SEQUENCE LISTING (20 sequences)

SEQ ID NO: 1 - protein, 5 aa, synthetic construct
SYSMN

SEQ ID NO: 2 - protein, 17 aa, synthetic construct
SISSSSSYIYYAESVKG

SEQ ID NO: 3 - protein, 9 aa, synthetic construct
VGLFGPFDI

SEQ ID NO: 4 - protein, 11 aa, synthetic construct
RASQSVSRYLG

SEQ ID NO: 5 - protein, 7 aa, synthetic construct
GASTRAT

SEQ ID NO: 6 - protein, 9 aa, synthetic construct
QQYGSSPWT

SEQ ID NO: 7 - protein, 118 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSS

SEQ ID NO: 8 - protein, 107 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIK

SEQ ID NO: 9 - protein, 447 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
SYGMH

SEQ ID NO: 12 - protein, 17 aa, synthetic construct
VIWYDGSNKYYADSVKG

SEQ ID NO: 13 - protein, 4 aa, synthetic construct
NGDH

SEQ ID NO: 14 - protein, 11 aa, synthetic construct
RASQSVSSNLA

SEQ ID NO: 15 - protein, 7 aa, synthetic construct
GASTRAT

SEQ ID NO: 16 - protein, 9 aa, synthetic construct
QQYNNWPRT

SEQ ID NO: 17 - protein, 113 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSS

SEQ ID NO: 18 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIK

SEQ ID NO: 19 - protein, 439 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0308257 A1
(43) Pub. Date: Oct. 8, 2026

(54) METHODS OF TREATING COLORECTAL CANCER USING A COMBINATION OF A CTLA-4 INHIBITOR AND A PD-1 INHIBITOR

(71) Applicant: Agenus Inc., Lexington, MA (US)

(72) Inventors: Joseph GROSSMAN, Lexington, MA (US); Dhan Sidhartha CHAND, Lexington, MA (US)

(21) Appl. No.: 19/547,215

(22) Filed: Feb. 23, 2026

Related U.S. Application Data
(63) Continuation of application No. PCT/US2024/043245, filed on Aug. 21, 2024.
(60) Provisional application No. 63/520,913, filed on Aug. 21, 2023.
(60) Provisional application No. 63/658,140, filed on Jun. 10, 2024.

(51) Int. Cl. C07K 16/28; A61K 9/00; A61K 39/00; A61P 35/00

(52) U.S. Cl. CPC C07K 16/2818; A61K 9/0019; A61P 35/00; A61K 2039/507; A61K 2039/54; A61K 2039/545; C07K 2317/41; C07K 2317/52; C07K 2317/565; C07K 2317/72

(57) ABSTRACT

Provided are methods for neoadjuvant treatment of colorectal cancer using a combination of a human CTLA-4 inhibitor and a human PD-1 inhibitor. These methods are particularly advantageous in that they can effectively treat non-MSI-H/dMMR colorectal cancers.

### RELATED APPLICATIONS

This application is a continuation of International Patent Application No. PCT/US2024/043245, filed Aug. 21, 2024, which claims priority to U.S. Provisional Patent Application Ser. No. 63/520,913, filed Aug. 21, 2023, and to U.S. Provisional Patent Application Ser. No. 63/658,140, filed Jun. 10, 2024, the entire disclosure of each of which is hereby incorporated herein by reference.

### REFERENCE TO SEQUENCE LISTING

This application contains a sequence listing which has been submitted electronically in ST.26 format and is hereby incorporated by reference in its entirety (said ST.26 copy, created on Aug. 21, 2024, is named “211852_seglist.xml” and is 19,908 bytes in size).

### BACKGROUND

Colorectal cancer is one of the most common cancers in the world and one of the leading causes of cancer-related mortality. Amongst various treatment options, one of the biggest advances for patients with colorectal cancer has been the advent of immunotherapy, in particular, inhibitors of death protein 1 (PD-1) and cytotoxic T lymphocyte-associated protein 4 (CTLA-4). Immunotherapy for colorectal cancer typically involves administration of anti-PD-1 or anti PD-L1 antibodies (e.g., pembrolizumab, nivolumab, durvalumab, and dostarlimab), either alone, or in combination with anti-CTLA-4 antibodies (e.g., ipilimumab and tremelimumab).

While immunotherapy can be useful for treatment of mismatch repair deficient/microsatellite-high (dMMR/MSI-H) colorectal cancer, it has shown less effectiveness in treating mismatch repair proficient/microsatellite stable (pMMR/MSS) colorectal cancer. Current treatments for pMMR/MSS colorectal cancer can include neoadjuvant chemotherapy, which comes with undesirable side effects.

Accordingly, there remains a need for novel, efficacious, and safe methods of treating colorectal cancer, including pMMR/MSS colorectal cancer. In particular, there is a need for immunotherapeutic neoadjuvant methods of treating pMMR/MSS colorectal cancer.

### SUMMARY

The instant disclosure is directed to methods for neoadjuvant treatment of colorectal cancer using a combination of a human CTLA-4 inhibitor and a human PD-1 inhibitor. These methods are particularly advantageous in that they can effectively treat pMMR/MSS colorectal cancers as well as dMMR/MSI-H colorectal cancers. As demonstrated in the Examples herein, these methods result in increased immune cell activation and a pattern of tumor regression that minimizes the chance of micro-metastatic disease being left behind following surgical resection and thus reduces the need for adjuvant chemotherapy.

Accordingly, in one aspect, the instant disclosure provides a method of treating a colorectal tumor in a subject in need thereof, the method comprising administering to the subject: a dose of a first antibody that specifically binds to human CTLA-4, wherein the first antibody comprises a human IgG heavy chain constant region that is a variant of a wild type human IgG heavy chain constant region, wherein the variant human IgG heavy chain constant region binds to FcγRIIIA with a higher affinity as compared to the affinity that the wild type human IgG heavy chain constant region binds to FcγRIIIA; and a first dose of a human PD-1 inhibitor, wherein the dose of the first antibody and the first dose of the human PD-1 inhibitor are each administered to the subject prior to surgical removal of the tumor.

In certain embodiments, the method further comprises administering to the subject: a second dose of the human PD-1 inhibitor, wherein the second dose is administered to the subject prior to surgical removal of the tumor.

In certain embodiments, the method further comprises administering to the subject: a third dose of the human PD-1 inhibitor; and a fourth dose of the human PD-1 inhibitor, wherein the fourth dose is administered to the subject prior to surgical removal of the tumor.

In certain embodiments, the first antibody comprises: a VH comprising the CDRH1, CDRH2, and CDRH3 amino acid sequences of the VH amino acid sequence set forth in SEQ ID NO: 7; and a VL comprising the CDRL1, CDRL2, and CDRL3 amino acid sequences of the VL amino acid sequence set forth in SEQ ID NO: 8.

In another aspect, the instant disclosure provides a method of treating a colorectal tumor in a subject in need thereof, the method comprising administering to the subject: a dose of a first antibody that specifically binds to human CTLA-4, wherein the first antibody comprises: a VH comprising the CDRH1, CDRH2, and CDRH3 amino acid sequences of the VH amino acid sequence set forth in SEQ ID NO: 7; and a VL comprising the CDRL1, CDRL2, and CDRL3 amino acid sequences of the VL amino acid sequence set forth in SEQ ID NO: 8; a first dose of a human PD-1 inhibitor; and a second dose of the human PD-1 inhibitor, wherein the dose of the first antibody and the first and second doses of the human PD-1 inhibitor are each administered to the subject prior to surgical removal of the tumor.

In certain embodiments, the subject does not receive a chemotherapeutic agent as part of the neoadjuvant treatment.

In certain embodiments, the first antibody comprises the CDRH1, CDRH2, CDRH3, CDRL1, CDRL2, and CDRL3 amino acid sequences set forth in SEQ ID NOs: 1, 2, 3, 4, 5, and 6, respectively. In certain embodiments, the first antibody comprises a VH comprising the amino acid sequence set forth in SEQ ID NO: 7 and a VL comprising the amino acid sequence set forth in SEQ ID NO: 8. In certain embodiments, the first antibody comprises a human IgG1 heavy chain constant region comprising S239D/A330L/1332E mutations, numbered according to the EU numbering system. In certain embodiments, the first antibody comprises a heavy chain comprising the amino acid sequence set forth in SEQ ID NO: 9 and a light chain comprising the amino acid sequence set forth in SEQ ID NO: 10. In certain embodiments, the first antibody is botensilimab. In certain embodiments, the first antibody is afucosylated.

In certain embodiments, the human PD-1 inhibitor is a second antibody that specifically binds to human PD-1 or human PD-L1. In certain embodiments, the second antibody comprises: a VH comprising the CDRH1, CDRH2, and CDRH3 amino acid sequences of the VH amino acid sequence set forth in SEQ ID NO: 17; and a VL comprising the CDRL1, CDRL2, and CDRL3 amino acid sequences of the VL amino acid sequence set forth in SEQ ID NO: 18. In certain embodiments, the second antibody comprises the CDRH1, CDRH2, CDRH3, CDRL1, CDRL2, and CDRL3 amino acid sequences set forth in SEQ ID NOs: 11, 12, 13, 14, 15, and 16, respectively. In certain embodiments, the second antibody comprises a VH comprising the amino acid sequence set forth in SEQ ID NO: 17 and a VL comprising the amino acid sequence set forth in SEQ ID NO: 18. In certain embodiments, the second antibody comprises a human IgG4 heavy chain constant region comprising an S228P mutation, numbered according to the EU numbering system. In certain embodiments, the second antibody comprises a heavy chain comprising the amino acid sequence set forth in SEQ ID NO: 19 and a light chain comprising the amino acid sequence set forth in SEQ ID NO: 20. In certain embodiments, the second antibody is balstilimab.

In certain embodiments, the human PD-1 inhibitor is selected from the group consisting of: adebrelimab, atezolizumab, avelumab, camrelizumab, cemiplimab, cosibelimab, dostarlimab, durvalumab, enlonstobart, envafolimab, nivolumab, pembrolizumab, penpulimab, pidilizumab, prolgolimab, pucotenlimab, retifanlimab, serplulimab, sintilimab, socazolimab, sugemalimab, tagitanlimab, tislelizumab, toripalimab, and zimberelimab.

In certain embodiments, the dose of the first antibody and the first dose of the human PD-1 inhibitor are administered on the same day. In certain embodiments, the dose of the first antibody and the first dose of the human PD-1 inhibitor are administered simultaneously. In certain embodiments, the dose of the first antibody is administered prior to the first dose of the human PD-1 inhibitor. In certain embodiments, the dose of the first antibody is administered after the first dose of the human PD-1 inhibitor.

In certain embodiments, the second dose of the human PD-1 inhibitor is administered 5 to 30 days, 7 to 21 days, 9 to 19 days, 12 to 19 days, or 14 days after the first dose of the human PD-1 inhibitor is administered. In certain embodiments, the second dose of the human PD-1 inhibitor is administered 14 days after the first dose of the human PD-1 inhibitor is administered.

In certain embodiments, the third dose of the human PD-1 inhibitor is administered 5 to 30 days, 7 to 21 days, 9 to 19 days, 12 to 19 days, or 14 days after the second dose of the human PD-1 inhibitor is administered. In certain embodiments, the third dose of the human PD-1 inhibitor is administered 14 days after the second dose of the human PD-1 inhibitor is administered.

In certain embodiments, the fourth dose of the human PD-1 inhibitor is administered 5 to 30 days, 7 to 21 days, 9 to 19 days, 12 to 19 days, or 14 days after the third dose of the human PD-1 inhibitor is administered. In certain embodiments, the fourth dose of the human PD-1 inhibitor is administered 14 days after the third dose of the human PD-1 inhibitor is administered.

In certain embodiments, the dose of the first antibody is 10 mg to 250 mg, optionally 25 mg to 200 mg, optionally 50 mg to 100 mg, optionally 75 mg. In certain embodiments, the dose of the first antibody is about 75 mg.

In certain embodiments, the first, second, third, and/or fourth dose of the human PD-1 inhibitor is 100 mg to 750 mg, 200 mg to 500 mg, or 240 mg. In certain embodiments, the first, second, third, and/or fourth dose of the human PD-1 inhibitor is about 240 mg. In certain embodiments, the first, second, third, and/or fourth dose of the human PD-1 inhibitor is about 450 mg.

In certain embodiments, the dose of the first antibody and/or the first dose of the human PD-1 inhibitor is administered 10 to 100 days, 14 to 98 days, 14 to 91 days, 14 to 84 days, 14 to 77 days, 14 to 70 days, 14 to 63 days, 14 to 56 days, 16 to 61 days, 19 to 61 days, or 21 to 56 days before surgical removal of the tumor. In certain embodiments, the dose of the first antibody and/or the first dose of the human PD-1 inhibitor is administered 28 days before surgical removal of the tumor. In certain embodiments, the dose of the first antibody and/or the first dose of the human PD-1 inhibitor is administered 56 days before surgical removal of the tumor.

In certain embodiments, the second dose of the human PD-1 inhibitor is administered at least 7 days before surgical removal of the tumor. In certain embodiments, the second dose of the human PD-1 inhibitor is administered 1 to 60 days, 5 to 45 days, or 7 to 42 days before surgical removal of the tumor. In certain embodiments, the second dose of the human PD-1 inhibitor is administered 14 days before surgical removal of the tumor.

In certain embodiments, the fourth dose of the human PD-1 inhibitor is administered at least 7 days before surgical removal of the tumor. In certain embodiments, the fourth dose of the human PD-1 inhibitor is administered 1 to 60 days, 5 to 45 days, or 7 to 42 days before surgical removal of the tumor. In certain embodiments, the fourth dose of the human PD-1 inhibitor is administered 14 days before surgical removal of the tumor.

In certain embodiments, the colorectal tumor is colorectal adenocarcinoma. In certain embodiments, the colorectal tumor is not metastatic. In certain embodiments, the colorectal tumor is a primary tumor. In certain embodiments, the subject has a RAS mutation. In certain embodiments, the RAS mutation is a KRAS or NRAS mutation. In certain embodiments, the colorectal tumor is microsatellite instable—high (MSI-H). In certain embodiments, the colorectal tumor is not microsatellite instable—high (MSI-H). In certain embodiments, the colorectal tumor is microsatellite stable (MSS). In certain embodiments, the colorectal tumor is mismatch repair deficient (dMMR). In certain embodiments, the colorectal tumor is not mismatch repair deficient (dMMR).

In certain embodiments, the first antibody and/or the human PD-1 inhibitor is administered intravenously. In certain embodiments, the first antibody and/or the human PD-1 inhibitor is administered by intravenous infusion over about 30 minutes.

In certain embodiments, the subject is at least 18 years of age. In certain embodiments, the subject has histologically, cytologically, or clinically confirmed adenocarcinoma of the colon. In certain embodiments, before administration of the first antibody and/or the human PD-1 inhibitor the subject has an Eastern Cooperative Oncology Group performance status of 0-2.

In certain embodiments, before administration of the first antibody and/or the human PD-1 inhibitor the subject has adequate organ and bone marrow reserve function as defined by one or more of: absolute neutrophil count≥1.5×10⁹ per L; platelets≥100×10⁹ per L; hemoglobin≥8.0 g/dL without a transfusion that has occurred within 2 weeks of hemoglobin measurement; creatinine clearance≥40 mL/min as measured or calculated per local institutional standards; aspartate aminotransferase≤2.5× upper limit of normal (ULN); alanine aminotransferase≤2.5×ULN; and total bilirubin≤1.5×ULN.

In certain embodiments, before administration of the first antibody and/or the human PD-1 inhibitor, the subject does not have metastases identified using standard of care radiographic imaging. In certain embodiments, before administration of the first antibody and/or the human PD-1 inhibitor, the subject is not pregnant and/or is not breastfeeding. In certain embodiments, the subject has not received a live vaccination within 28 days prior to administration of the first antibody and/or the human PD-1 inhibitor. In certain embodiments, before administration of the first antibody and/or the human PD-1 inhibitor, the subject does not have clinically significant cardiovascular disease or an active infection requiring treatment. In certain embodiments, the subject has not received systemic corticosteroid therapy within 7 days prior to administration of the first antibody and/or the human PD-1 inhibitor.

In certain embodiments, administration of the first antibody and the human PD-1 inhibitor reduces residual viable tumor cells in the subject following surgical removal of the tumor. In certain embodiments, administration of the first antibody and the human PD-1 inhibitor reduces minimal residual disease in the subject following surgical removal of the tumor. In certain embodiments, minimal residual disease is assessed via measurement of circulating tumor DNA. In certain embodiments, administration of the first antibody and the human PD-1 inhibitor reduces the size of the tumor in the subject.

In certain embodiments, administration of the first antibody and the human PD-1 inhibitor increases T-cell, memory T-cell, myeloid cell, and/or antigen presenting cell activation in the subject. In certain embodiments, administration of the first antibody and the human PD-1 inhibitor reduces the number of Treg cells in the subject.

In an aspect, provided herein is an antibody that specifically binds to human CTLA-4 and a human PD-1 inhibitor for use in the treatment of a colorectal tumor, wherein the treatment is performed according to the method of any one of the preceding claims.

In an aspect, provided herein is an antibody that specifically binds to human CTLA-4 and a human PD-1 inhibitor for use in the manufacture of a medicament for the treatment of a colorectal tumor, wherein the treatment is performed according to the method of any one of the preceding claims.

In an aspect, provided herein is an antibody that specifically binds to human CTLA-4 and a human PD-1 inhibitor for the treatment of a colorectal tumor, wherein the treatment is performed according to the method of any one of the previous claims.

### BRIEF DESCRIPTION OF THE DRAWINGS

FIG. 1 is a waterfall plot showing the pathologic response as an output of histopathological tumor regression (%) in patients with microsatellite stable (MSS; n=9) and microsatellite instability-high (MSI-High; n=3) colon and rectal cancers upon neoadjuvant bot/bal treatment, according to aspects of this disclosure. * and dotted borders: patients with rectal cancer (NEST-IDs 7, 2, 3 and 9). Sex of patient is indicated as male (M) or female (F).

FIG. 2A is a listing of pre- and post-treatment stage, pathological response, and adverse events, and FIG. 2B is a swimmer's plot showing the follow-up to date and circulating tumor DNA (ctDNA) status (solid black circles—ctDNA-positive, hollow white circles—ctDNA-negative) in patients with resectable colon cancer that received neoadjuvant bot/bal treatment, according to aspects of this disclosure. * indicates patients with rectal cancer (NEST-IDs 7, 2, 3, and 9). Sex of patient is indicated as male (M) or female (F), and pre- and post-treatment stage is indicated according to the American Joint Committee on Cancer staging system for colorectal cancer. Grade of adverse events experienced by individual patients is also indicated, assessed according to the Common Terminology Criteria for Adverse Events (CTCAE) v5.0. In FIG. 2B, the first shaded oval starting at 0 months represents the treatment with bot/bal combination immunotherapy followed by the additional dose of bal. The second shaded oval represents the time from the dose of bal to surgery (indicated by triangle).

FIGS. 3A and 3B are plots showing changes in CD68 and CD3 (FIG. 3A), and CD4 and CD8 (FIG. 3B) in patients with resectable colon cancer that received neoadjuvant bot/bal treatment, according to aspects of this disclosure. In FIG. 3A, fold change in CD68 (left bar for each patient) and CD3 (right bar for each patient) is shown. In FIG. 3B, fold change in CD4 (left bar for each patient) and CD8 (right bar for each patient) is shown. In both plots, the x-axis indicates each individual patient's corresponding NEST-ID. Data of patients is presented grouped by patients with microsatellite stable (MSS) colorectal cancer that had ≥50% response, MSS<50% response, and microsatellite instable-high (MSI High).

FIG. 4 is a plot showing circulating tumor DNA (ctDNA) kinetics/decline (%) in patients who had pre-operative testing on immunotherapy that received neoadjuvant bot/bal treatment, according to aspects of this disclosure. Numbers in boxes indicate the number of days into bot/bal immunotherapy followed by ctDNA decline (%).

FIGS. 5A-5F are a series of pie charts showing mutations noted in the cohort of patients with resectable colon cancer that received neoadjuvant bot/bal treatment, according to aspects of this disclosure. Specific mutations are indicated for TP53 (FIG. 5A), APC (FIG. 5B), KRAS (FIG. 5C), CTNNBI (FIG. 5D), PIK3CA (FIG. 5E), and BRAF (FIG. 5F).

FIG. 6 is a plot showing the changes in peripheral lymphocytes (%) 2 weeks post bot/bal immunotherapy, according to aspects of this disclosure. Patient NEST-ID and sex (male [M] and female [F]) is indicated and grouped according to having MSS or MSI-High colorectal cancer.

FIG. 7 is a plot showing changes in neutrophil-to-lymphocyte-ratio (NLR %) 2 weeks post-bot/bal immunotherapy, according to aspects of this disclosure. Patient NEST-ID and sex (male [M] and female [F]) is indicated and grouped according to having MSS or MSI-High colorectal cancer.

FIG. 8 is a waterfall plot showing the pathologic response as an output of histopathological tumor regression (%) in patients with microsatellite stable (MSS; n=17) and microsatellite instability-high (MSI-High; n=3) colorectal cancer upon treatment with neoadjuvant bot (1 dose) and bal (2 or 4 doses), according to aspects of this disclosure.

FIGS. 9A-9D are plots showing changes in immune cell populations in a first patient with resectable colorectal cancer upon neoadjuvant treatment using a combination of botensilimab and balstilimab, according to aspects of this disclosure. The densities of B cells (FIG. 9A) and T-cells (FIG. 9B), the proportion of Th cells that are Treg (FIG. 9C), and the proportion of immune cells that are proliferating (FIG. 9D) are shown in a pre-treatment biopsy sample (left bars) and in a resection tumor sample (right bars).

FIGS. 10A-10D are plots showing changes in immune cell populations in a second patient with resectable colorectal cancer upon neoadjuvant treatment using a combination of botensilimab and balstilimab, according to aspects of this disclosure. The densities of B cells (FIG. 10A) and T-cells (FIG. 10B), the proportion of Th cells that are Treg (FIG. 10C), and the proportion of immune cells that are proliferating (FIG. 10D) are shown in a pre-treatment biopsy sample (left bars), in a resection tumor sample area associated with tumor regression (tumor area #1; middle bars) and in a resection tumor sample area with no obvious tumor regression (tumor area #2; right bars).

### DETAILED DESCRIPTION

The instant disclosure is directed to methods for neoadjuvant treatment of colorectal cancer using a combination of a human CTLA-4 inhibitor and a human PD-1 inhibitor. These methods are particularly advantageous in that they can effectively treat non-MSI-H/dMMR colorectal cancers. As demonstrated in the Examples herein, these methods result in increased immune cell activation and a pattern of tumor regression that minimizes the chance of micro-metastatic disease being left behind following surgical resection and thus reduces the need for adjuvant chemotherapy.

### Definitions

As used herein, the term “CTLA-4” refers to cytotoxic T-lymphocyte-associated protein 4. As used herein, the term “human CTLA-4” refers to a human CTLA-4 protein encoded by a wild-type human CTLA-4 gene, e.g., RefSeq accession number NM_005214.5 or NM_001037631.2. An exemplary immature amino acid sequence of human CTLA-4 is set forth in RefSeq accession number NP_005205.2.

As used herein, the term “PD-1” refers to the programmed cell death protein 1. As used herein, the term “human PD-1” refers to a human PD-1 protein encoded by a wild-type human PD-1 gene, e.g., RefSeq accession number NM_005018.3. An exemplary immature amino acid sequence of human PD-1 is provided as in RefSeq accession number NP_005009.2.

As used herein, the term “PD-L1” refers to the programmed cell death ligand 1. As used herein, the term “human PD-L1” refers to a human PD-L1 protein encoded by a wild-type human PD-L1 gene, e.g., RefSeq accession number NM_014143.4. An exemplary immature amino acid sequence of human PD-L1 is provided as in RefSeq accession number NP_054862.1.

As used herein, the terms “antibody” and “antibodies” include full-length antibody molecules, antigen-binding fragments of full-length antibody molecules, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG1, IgG2, IgG3, IgG4, IgA1, or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. In certain embodiments, the antibody is a human antibody.

As used herein, the term “CDR” or “complementarity determining region” means the noncontiguous antigen combining sites found within the variable regions of heavy and light chain polypeptides. In certain embodiments, CDRs of an antibody disclosed herein are determined according to Kabat et at, J Biot. Chem. 252, 6609-6616 (1977) and Kabat et al, Sequences of protein of immunological interest (1991), each of which is herein incorporated by reference in its entirely. In certain embodiments, CDRs of an antibody disclosed herein are determined according to the Chothia numbering scheme (see, e.g., Chothia C & Lesk AM, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226, all of which are herein incorporated by reference in their entireties). In certain embodiments, CDRs of an antibody disclosed herein are determined according to MacCallum R M et al., (1996) J Mol Biol 262: 732-745, herein incorporated by reference in its entirety. See also, e.g., Martin A. “Protein Sequence and Structure Analysis of Antibody Variable Domains,” in Antibody Engineering, Kontermann and Dribel, eds., Chapter 31, pp. 422-439, Springer-Verlag, Berlin (2001), herein incorporated by reference in its entirety. In certain embodiments, CDRs of an antibody disclosed herein are determined according to the IMGT numbering system as described in: Lefranc M-P, (1999) The Immunologist 7: 132-136; Lefranc M-P et al, (1999) Nucleic Acids Res 27: 209-212, each of which is herein incorporated by reference in its entirety; and Lefranc M-P et al, (2009) Nucleic Acids Res 37: D1006-D1012. In certain embodiments, CDRs of an antibody disclosed herein are determined according to the AbM numbering scheme, which refers to AbM hypervariable regions, which represent a compromise between the Kabat CDRs and Chothia structural loops and are used by Oxford Molecular's AbM antibody modeling software (Oxford Molecular Group, Inc.), herein incorporated by reference in its entirety. In certain embodiments, CDRs of an antibody disclosed herein are determined according to the AHo numbering system, as described in Honegger and Pluckthun, J. Mol. Biol. 309:657-670 (2001), herein incorporated by reference in its entirety. In certain embodiments, CDRs of an antibody disclosed herein are each independently determined according to one of the Kabat, Chothia, MacCallum, IMGT, AHo, or AbM numbering schemes, or by structural analysis of the multispecific molecule, wherein the structural analysis identifies residues in the variable region(s) predicted to make contact with an epitope. CDRH1, CDRH2, and CDRH3 denote the heavy chain CDRs, and CDRL1, CDRL2, and CDRL3 denote the light chain CDRs.

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen, but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ϵ), gamma (γ), and mu (μ), based on the amino acid sequence of the constant region, which give rise to IgA, IgD, IgE, IgG, and IgM classes of antibodies, respectively, including subclasses of IgG, e.g., IgG1, IgG2, IgG3, and IgG4.

As used herein, the term “light chain” when used in reference to an antibody can refer to any distinct type, e.g., kappa (κ) or lambda (λ), based on the amino acid sequence of the constant region. Light chain amino acid sequences are well known in the art. In certain embodiments, the light chain is a human light chain.

As used herein, the term “specifically binds” refers to the specificity of an antibody for an antigen, as is understood by one skilled in the art. Binding molecules that specifically bind to an antigen typically bind to the antigen with an equilibrium dissociation constant (KD) of less than 1×10⁶ M, as measured by, e.g., ELISA assay, surface plasmon resonance, or other suitable assays known in the art. The skilled worker will appreciate that, in certain embodiments, a binding molecule can specifically bind to different antigens, e.g., different antigens that share a common epitope that is recognized by the binding molecule.

As used herein, the term “afucosylated” in the context of an Fc refers to a substantial lack of a fucose covalently attached, directly or indirectly, to residue 297 of the human IgG₁ Fc region, numbered according to the EU numbering system, or the corresponding residue in non-IgG₁ or non-human IgG₁ immunoglobulins. Thus, in a composition comprising a plurality of afucosylated antibodies, at least 70% of the antibodies will not be fucosylated, directly or indirectly (e.g., via intervening sugars) at residue 297 of the Fc region of the antibodies, and in some embodiments at least 80%, 85%, 90%, 95%, or 99% will not be fucosylated, directly or indirectly, at residue 297 of the Fc region.

As used herein, the term “CTLA-4 inhibitor” refers to a molecule that can inhibit the binding of CTLA-4 to its ligand, Cluster of differentiation 80 (CD80).

As used herein, the term “PD-1 inhibitor” refers to a molecule that can inhibit the binding of PD-1 to its ligand, Programmed death-ligand 1 (PD-L1).

As used herein, the term “EU numbering system” refers to the EU numbering convention for the constant regions of an antibody, as described in Edelman G. M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety.

As used herein, the term “subject” includes any human or non-human animal. In certain embodiments, the subject is a human.

As used herein, the term “effective amount” in the context of the administration of a therapy to a subject refers to the amount of a therapy that achieves a desired prophylactic or therapeutic effect.

As used herein, the term “Eastern Cooperative Oncology Group performance status” refers to the grade on the Eastern Cooperative Oncology Group Performance Status Scale determined for a subject prior to treatment. The Eastern Cooperative Oncology Group Performance Status Scale is well known in the art and describes a patient's level of function in terms of ability to care for themself, daily activity, and physical ability (walking, working, etc.).

As used herein, the term “about” when referring to a measurable value, such as a dosage, refers to variations of 10% of a given value or range, as are appropriate to perform the methods disclosed herein.

### CTLA-4 Inhibitors

Human CTLA-4 inhibitors that are useful in the methods and uses described herein include, but are not limited to, those described below.

In certain embodiments, the human CTLA-4 inhibitor is an antibody that specifically binds to human CTLA-4. In certain embodiments, the antibody that specifically binds to human CTLA-4 comprises: a heavy chain variable region (VH) comprising the CDRH1, CDRH2, and CDRH3 amino acid sequences of the VH amino acid sequence set forth in SEQ ID NO: 7. In certain embodiments, the antibody comprises a heavy chain variable region (VH) comprising the CDRH1, CDRH2, and CDRH3 amino acid sequences of the VH amino acid sequence set forth in SEQ ID NO: 7 and a light chain variable region (VL) comprising the CDRL1, CDRL2, and CDRL3 amino acid sequences of the VL amino acid sequence set forth in SEQ ID NO: 8.

In certain embodiments, the antibody that specifically binds to human CTLA-4 comprises the CDRH1, CDRH2, and CDRH3 amino acid sequences set forth in SEQ ID NOs: 1, 2, and 3, respectively. In certain embodiments, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In certain embodiments, the antibody that specifically binds to human CTLA-4 comprises a VH comprising an amino acid sequence which is at least 90%, 95%, 96%, 97%, 98%, 99%, or 100% identical to the amino acid sequence set forth in SEQ ID NO: 7. In certain embodiments, the antibody comprises a VH comprising the amino acid sequence set forth in SEQ ID NO: 7.

In certain embodiments, the antibody that specifically binds to human CTLA-4 comprises a VL comprising an amino acid sequence which is at least 90%, 95%, 96%, 97%, 98%, 99%, or 100% identical to the amino acid sequence set forth in SEQ ID NO: 8. In certain embodiments, the antibody comprises a VL comprising the amino acid sequence set forth in SEQ ID NO: 8.

In certain embodiments, the antibody that specifically binds to human CTLA-4 comprises: a VH comprising an amino acid sequence which is at least 90%, 95%, 96%, 97%, 98%, 99%, or 100% identical to the amino acid sequence set forth in SEQ ID NO: 7; and a VL comprising an amino acid sequence which is at least 90%, 95%, 96%, 97%, 98%, 99%, or 100% identical to the amino acid sequence set forth in SEQ ID NO: 8. In certain embodiments, the antibody comprises a VH comprising the amino acid sequence set forth in SEQ ID NO: 7 and a VL comprising the amino acid sequence set forth in SEQ ID NO: 8.

In certain embodiments, the antibody that specifically binds to human CTLA-4 comprises a heavy chain constant region selected from the group consisting of human IgG1, IgG2, IgG3, IgG4, IgA1, and IgA2. In certain embodiments, the heavy chain constant region is IgG1. In certain embodiments, the heavy chain constant region is IgG2. In certain embodiments, the antibody that specifically binds to human CTLA-4 comprises an IgG₁ heavy chain constant region.

In certain embodiments, the antibody comprises a light chain constant region selected from the group consisting of a human kappa light chain constant region and a human lambda light chain constant region.

In certain embodiments, the antibody that specifically binds to human CTLA-4 comprises a human IgG heavy chain constant region that is a variant of a wild type human IgG heavy chain constant region, wherein the variant human IgG heavy chain constant region binds to FcγRIIIA with a higher affinity than the wild type human IgG heavy chain constant region binds to FcγRIIIA.

In certain embodiments, the IgG region of the antibody that specifically binds to human CTLA-4 has an increased affinity for FcγRIIIA, e.g., as compared with an antibody with a wild-type Fc region, e.g., an IgG₁ Fc. Sequence alterations that result in increased affinity for FcγRIIIA are known in the art, for example, in Kellner et al., Methods 65: 105-113 (2014), Lazar et al., Proc Natl Acad Sci 103: 4005-4010 (2006), Shields et al., J Biol Chem. 276(9):6591-6604 (2001), each of which is herein incorporated by reference in its entirety.

In certain embodiments, the antibody that specifically binds to human CTLA-4 comprises a heavy chain constant region, e.g., an IgG₁ constant region, or fragment thereof comprising a mutation selected from the group consisting of: L235V, G236A, S239D, F243L, T256A, K290A, R292P, S298A, Y300L, V305I, A330L, 1332E, E333A, K334A, A339T, and P396L, and combinations thereof, numbered according to the EU numbering system.

In certain embodiments, the antibody that specifically binds to human CTLA-4 comprises a heavy chain constant region, e.g., an IgG1 constant region, or fragment thereof comprising S239D, numbered according to the EU numbering system. In certain embodiments, the antibody that specifically binds to human CTLA-4 comprises a heavy chain constant region, e.g., an IgG1 constant region, or fragment thereof comprising T256A, numbered according to the EU numbering system. In certain embodiments, the antibody that specifically binds to human CTLA-4 comprises a heavy chain constant region, e.g., an IgG1 constant region, or fragment thereof comprising K290A, numbered according to the EU numbering system. In certain embodiments, the antibody that specifically binds to human CTLA-4 comprises a heavy chain constant region, e.g., an IgG1 constant region, or fragment thereof comprising S298A, numbered according to the EU numbering system. In certain embodiments, the antibody that specifically binds to human CTLA-4 comprises a heavy chain constant region, e.g., an IgG1 constant region, or fragment thereof comprising 1332E, numbered according to the EU numbering system. In certain embodiments, the antibody that specifically binds to human CTLA-4 comprises a heavy chain constant region, e.g., an IgG1 constant region, or fragment thereof comprising E333A, numbered according to the EU numbering system. In certain embodiments, the antibody that specifically binds to human CTLA-4 comprises a heavy chain constant region, e.g., an IgG1 constant region, or fragment thereof comprising K334A, numbered according to the EU numbering system. In certain embodiments, the antibody that specifically binds to human CTLA-4 comprises a heavy chain constant region, e.g., an IgG1 constant region, or fragment thereof comprising A339T, numbered according to the EU numbering system. In certain embodiments, the antibody that specifically binds to human CTLA-4 comprises a heavy chain constant region, e.g., an IgG1 constant region, or fragment thereof comprising S239D and 1332E, numbered according to the EU numbering system. In certain embodiments, the antibody that specifically binds to human CTLA-4 comprises an IgG₁ heavy chain constant region that comprises S239D/I332E mutations, numbered according to the EU numbering system. In certain embodiments, the antibody that specifically binds to human CTLA-4 comprises a heavy chain constant region, e.g., an IgG1 constant region, or fragment thereof comprising S239D, A330L, and 1332E, numbered according to the EU numbering system. In certain embodiments, the antibody that specifically binds to human CTLA-4 comprises an IgG₁ heavy chain constant region that comprises S239D/A330L/1332E mutations, numbered according to the EU numbering system. In certain embodiments, the antibody that specifically binds to human CTLA-4 comprises a heavy chain constant region, e.g., an IgG1 constant region, or fragment thereof comprising S298A, E333A, and K334A, numbered according to the EU numbering system. In certain embodiments, the antibody that specifically binds to human CTLA-4 comprises a heavy chain constant region, e.g., an IgG1 constant region, or fragment thereof comprising G236A, S239D, and 1332E, numbered according to the EU numbering system. In certain embodiments, the antibody that specifically binds to human CTLA-4 comprises a heavy chain constant region, e.g., an IgG1 constant region, or fragment thereof comprising F243L, R292P, Y300L, V305I, and P396L, numbered according to the EU numbering system. In certain embodiments, the antibody that specifically binds to human CTLA-4 comprises an IgG₁ heavy chain constant region that comprises L235V/F243L/R292P/Y300L/P396L mutations, numbered according to the EU numbering system.

In certain embodiments, the antibody that specifically binds to human CTLA-4 comprises a heavy chain comprising an amino acid sequence which is at least 90%, 95%, 96%, 97%, 98%, 99%, or 100% identical to the amino acid sequence set forth in SEQ ID NO: 9. In certain embodiments, the antibody comprises a heavy chain comprising the amino acid sequence set forth in SEQ ID NO: 9.

In certain embodiments, the antibody that specifically binds to human CTLA-4 comprises a light chain comprising an amino acid sequence which is at least 90%, 95%, 96%, 97%, 98%, 99%, or 100% identical to the amino acid sequence set forth in SEQ ID NO: 10. In certain embodiments, the antibody comprises a light chain comprising the amino acid sequence set forth in SEQ ID NO: 10.

In certain embodiments, the antibody that specifically binds to human CTLA-4 comprises a heavy chain comprising an amino acid sequence which is at least 90%, 95%, 96%, 97%, 98%, 99%, or 100% identical to the amino acid sequence set forth in SEQ ID NO: 9; and a light chain comprising an amino acid sequence which is at least 90%, 95%, 96%, 97%, 98%, 99%, or 100% identical to the amino acid sequence set forth in SEQ ID NO: 10. In certain embodiments, the antibody comprises a heavy chain comprising the amino acid sequence set forth in SEQ ID NO: 9 and a light chain comprising the amino acid sequence set forth in SEQ ID NO: 10. In certain embodiments, the amino acid sequence of the heavy chain consists of the amino acid sequence set forth in SEQ ID NO: 9 and the amino acid sequence of the light chain consists of the amino acid sequence set forth in SEQ ID NO: 10.

In certain embodiments, the antibody that specifically binds to human CTLA-4 is afucosylated.

In certain embodiments the antibody that specifically binds to human CTLA-4 is botensilimab.

The amino acid sequences of exemplary anti-CTLA-4 antibodies are provided in Table 1 herein.

TABLE 1
Amino acid sequences of exemplary anti-CTLA-4 antibodies.

| Description | SEQ ID NO: | Amino acid sequence |
|---|---|---|
| CDRH1 | 1 | SYSMN |
| CDRH2 | 2 | SISSSSSYIYYAESVKG |
| CDRH3 | 3 | VGLFGPFDI |
| CDRL1 | 4 | RASQSVSRYLG |
| CDRL2 | 5 | GASTRAT |
| CDRL3 | 6 | QQYGSSPWT |
| VH | 7 | EVQLVESGGGLVKPGGSLRLSCAASGFTFSSYSMNWVR |
| | | QAPGKGLEWVSSISSSSSYIYYAESVKGRFTISRDNAKN |
| | | SLYLQMNSLRAEDTAVYYCARVGLFGPFDIWGQGTLV |
| | | TVSS |
| VL | 8 | EIVLTQSPGTLSLSPGERATLSCRASQSVSRYLGWYQQK |
| | | PGQAPRLLIYGASTRATGIPDRFSGSGSGTDFTLTITRL |
| | | EPEDFAVYYCQQYGSSPWTFGQGTKVEIK |
| Heavy chain | 9 | EVQLVESGGGLVKPGGSLRLSCAASGFTFSSYSMNWVR |
| | | QAPGKGLEWVSSISSSSSYIYYAESVKGRFTISRDNAKN |
| | | SLYLQMNSLRAEDTAVYYCARVGLFGPFDIWGQGTLV |
| | | TVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPC |
| | | PAPELLGGPDVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALPLPEEKTISKAKGQP |
| | | REPQVYTLPPSREEMTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPG |
| Light chain | 10 | EIVLTQSPGTLSLSPGERATLSCRASQSVSRYLGWYQQK |
| | | PGQAPRLLIYGASTRATGIPDRFSGSGSGTDFTLTITRLEP |
| | | EDFAVYYCQQYGSSPWTFGQGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYAC |
| | | EVTHQGLSSPVTKSFNRGEC |

### PD-1 Inhibitors

Human PD-1 inhibitors that are useful in the methods and uses described herein include but are not limited to those described below.

In certain embodiments, the human PD-1 inhibitor is an antibody that specifically binds to human PD-1. In certain embodiments, the antibody that specifically binds to human PD-1 comprises: a heavy chain variable region (VH) comprising the CDRH1, CDRH2, and CDRH3 amino acid sequences of the VH amino acid sequence set forth in SEQ ID NO: 17. In certain embodiments, the antibody that specifically binds to human PD-1 comprises: a light chain variable region (VL) comprising the CDRL1, CDRL2, and CDRL3 amino acid sequences of the VL amino acid sequence set forth in SEQ ID NO: 18. In certain embodiments, the antibody that specifically binds to human PD-1 comprises a heavy chain variable region (VH) comprising the CDRH1, CDRH2, and CDRH3 amino acid sequences of the VH amino acid sequence set forth in SEQ ID NO: 17 and a light chain variable region (VL) comprising the CDRL1, CDRL2, and CDRL3 amino acid sequences of the VL amino acid sequence set forth in SEQ ID NO: 18.

In certain embodiments, the antibody that specifically binds to human PD-1 comprises the CDRH1, CDRH2, and CDRH3 amino acid sequences set forth in SEQ ID NO: 11, 12, and 13, respectively. In certain embodiments, the antibody that specifically binds to human PD-1 comprises the CDRL1, CDRL2, and CDRL3 amino acid sequences set forth in SEQ ID NO: 14, 15, and 16, respectively. In certain embodiments, the antibody that specifically binds to human PD-1 comprises the CDRH1, CDRH2, CDRH3, CDRL1, CDRL2, and CDRL3 amino acid sequences set forth in SEQ ID NOs: 11, 12, 13, 14, 15, and 16, respectively.

In certain embodiments, the antibody that specifically binds to human PD-1 comprises a VH comprising an amino acid sequence which is at least 90%, 95%, 96%, 97%, 98%, 99%, or 100% identical to the amino acid sequence set forth in SEQ ID NO: 17. In certain embodiments, the antibody that specifically binds to human PD-1 comprises a VH comprising the amino acid sequence set forth in SEQ ID NO: 17.

In certain embodiments, the antibody that specifically binds to human PD-1 comprises a VL comprising an amino acid sequence which is at least 90%, 95%, 96%, 97%, 98%, 99%, or 100% identical to the amino acid sequence set forth in SEQ ID NO: 18. In certain embodiments, the antibody that specifically binds to human PD-1 comprises a VL comprising the amino acid sequence set forth in SEQ ID NO: 18.

In certain embodiments, the antibody that specifically binds to human PD-1 comprises: a VH comprising an amino acid sequence which is at least 90%, 95%, 96%, 97%, 98%, 99%, or 100% identical to the amino acid sequence set forth in SEQ ID NO: 17; and a VL comprising an amino acid sequence which is at least 90%, 95%, 96%, 97%, 98%, 99%, or 100% identical to the amino acid sequence set forth in SEQ ID NO: 18. In certain embodiments, the antibody that specifically binds to human PD-1 comprises a VH comprising the amino acid sequence set forth in SEQ ID NO: 17; and a VL comprising the amino acid sequence set forth in SEQ ID NO: 18.

In certain embodiments, the antibody that specifically binds to human PD-1 comprises a heavy chain constant region selected from the group consisting of human IgG1, IgG2, IgG3, IgG4, IgA1, and IgA2. In certain embodiments, the heavy chain constant region is IgG1. In certain embodiments, the heavy chain constant region is IgG2. In certain embodiments, the antibody comprises a light chain constant region selected from the group consisting of a human kappa light chain constant region and a human lambda light chain constant region.

In certain embodiments, the antibody that specifically binds to human PD-1 comprises an IgG4 heavy chain constant region. In certain embodiments, the amino acid sequence of the IgG4 heavy chain constant region comprises an S228P mutation, numbered according to the EU numbering system.

In certain embodiments, the antibody that specifically binds to human PD-1 comprises a heavy chain comprising an amino acid sequence which is at least 90%, 95%, 96%, 97%, 98%, 99%, or 100% identical to the amino acid sequence set forth in SEQ ID NO: 19. In certain embodiments, the antibody comprises a heavy chain comprising the amino acid sequence set forth in SEQ ID NO: 19.

In certain embodiments, the antibody that specifically binds to human PD-1 comprises a light chain comprising an amino acid sequence which is at least 90%, 95%, 96%, 97%, 98%, 99%, or 100% identical to the amino acid sequence set forth in SEQ ID NO: 20. In certain embodiments, the antibody comprises a light chain comprising the amino acid sequence set forth in SEQ ID NO: 20.

In certain embodiments, the antibody that specifically binds to human PD-1 comprises a heavy chain comprising an amino acid sequence which is at least 90%, 95%, 96%, 97%, 98%, 99%, or 100% identical to the amino acid sequence set forth in SEQ ID NO: 19; and a light chain comprising an amino acid sequence which is at least 90%, 95%, 96%, 97%, 98%, 99%, or 100% identical to the amino acid sequence set forth in SEQ ID NO: 20. In certain embodiments, the antibody that specifically binds to human PD-1 comprises a heavy chain comprising the amino acid sequence set forth in SEQ ID NO: 19 and a light chain comprising the amino acid sequence set forth in SEQ ID NO: 20. In certain embodiments, the amino acid sequence of the heavy chain consists of the amino acid sequence set forth in SEQ ID NO: 19 and the amino acid sequence of the light chain consists of the amino acid sequence set forth in SEQ ID NO: 20.

In certain embodiments the antibody that specifically binds to human PD-1 is balstilimab.

The amino acid sequences of exemplary anti-PD-1 antibodies are provided in Table 2 herein.

TABLE 2
Amino acid sequences of exemplary anti-PD-1 antibodies.

| Description | SEQ ID NO: | Amino acid sequence |
|---|---|---|
| CDRH1 | 11 | SYGMH |
| CDRH2 | 12 | VIWYDGSNKYYADSVKG |
| CDRH3 | 13 | NGDH |
| CDRL1 | 14 | RASQSVSSNLA |
| CDRL2 | 15 | GASTRAT |
| CDRL3 | 16 | QQYNNWPRT |
| VH | 17 | QVQLVESGGGVVQPGRSLRLSCAASGFTFSSYGMHWV |
| | | RQAPGKGLEWVAVIWYDGSNKYYADSVKGRFTISRD |
| | | NSKNTLYLQMNSLRAEDTAVYYCASNGDHWGQGTLV |
| | | TVSS |
| VL | 18 | EIVMTQSPATLSVSPGERATLSCRASQSVSSNLAWYQQ |
| | | KPGQAPRLLIYGASTRATGIPARFSGSGSGTEFTLTIS |
| | | SLQSEDFAVYYCQQYNNWPRTFGQGTKVEIK |
| Heavy chain | 19 | QVQLVESGGGVVQPGRSLRLSCAASGFTFSSYGMHWV |
| | | RQAPGKGLEWVAVIWYDGSNKYYADSVKGRFTISRD |
| | | NSKNTLYLQMNSLRAEDTAVYYCASNGDHWGQGTLV |
| | | TVSSASTKGPSVFPLAPCSRSTSESTAALGCLVKDYFPE |
| | | PVTVSWNSGALTSGVHTFPAVLQSSGLYSLSSVVTVPS |
| | | SSLGTKTYTCNVDHKPSNTKVDKRVESKYGPPCPPCPA |
| | | PEFLGGPSVFLFPPKPKDTLMISRTPEVTCVVVDVSQED |
| | | PEVQFNWYVDGVEVHNAKTKPREEQFNSTYRVVSVLT |
| | | VLHQDWLNGKEYKCKVSNKGLPSSIEKTISKAKGQPR |
| | | EPQVYTLPPSQEEMTKNQVSLTCLVKGFYPSDIAVEWE |
| | | SNGQPENNYKTTPPVLDSDGSFFLYSRLTVDKSRWQE |
| | | GNVFSCSVMHEALHNHYTQKSLSLSLG |
| Light chain | 20 | EIVMTQSPATLSVSPGERATLSCRASQSVSSNLAWYQQ |
| | | KPGQAPRLLIYGASTRATGIPARFSGSGSGTEFTLTISSL |
| | | QSEDFAVYYCQQYNNWPRTFGQGTKVEIKRTVAAPSV |
| | | FIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |

In certain embodiments the antibody that specifically binds to human PD-1 or human PD-L1 is adebrelimab, atezolizumab, avelumab, camrelizumab, cemiplimab, cosibelimab, dostarlimab, durvalumab, enlonstobart, envafolimab, nivolumab, pembrolizumab, penpulimab, pidilizumab, prolgolimab, pucotenlimab, retifanlimab, serplulimab, sintilimab, socazolimab, sugemalimab, tagitanlimab, tislelizumab, toripalimab, and zimberelimab.

Further non-limiting examples of anti-PD-1 antibodies that may be used in treatment methods described herein are disclosed in the following patents and patent applications, which are incorporated herein by reference in their entireties for all purposes: U.S. Pat. Nos. 6,808,710; 7,332,582; 7,488,802; 8,008,449; 8,114,845; 8,168,757; 8,354,509; 8,686,119; 8,735,553; 8,747,847; 8,779,105; 8,927,697; 8,993,731; 9,102,727; 9,205,148; U.S. Publication No. US 2013/0202623 A1; U.S. Publication No. US 2013/0291136 A1; U.S. Publication No. US 2014/0044738 A1; U.S. Publication No. US 2014/0356363 A1; U.S. Publication No. US 2016/0075783 A1; and PCT Publication No. WO 2013/033091 A1; PCT Publication No. WO 2015/036394 A1; PCT Publication No. WO 2014/179664 A2; PCT Publication No. WO 2014/209804 A1; PCT Publication No. WO 2014/206107 A1; PCT Publication No. WO 2015/058573 A1; PCT Publication No. WO 2015/085847 A1; PCT Publication No. WO 2015/200119 A1; PCT Publication No. WO 2016/015685 A1; and PCT Publication No. WO 2016/020856 A1.

Further examples of anti-PD-L1 antibodies that may be used in treatment methods described herein are disclosed in the following patents and patent applications, which are incorporated herein by reference in their entireties for all purposes: U.S. Pat. Nos. 7,943,743; 8,168,179; 8,217,149; 8,552,154; 8,779,108; 8,981,063; 9,175,082; U.S.

Publication No. US 2010/0203056 A1; U.S. Publication No. US 2003/0232323 A1; U.S. Publication No. US 2013/0323249 A1; U.S. Publication No. US 2014/0341917 A1; U.S. Publication No. US 2014/0044738 A1; U.S. Publication No. US 2015/0203580 A1; U.S. Publication No. US 2015/0225483 A1; U.S. Publication No. US 2015/0346208 A1; U.S. Publication No. US 2015/0355184 A1; and PCT Publication No. WO 2014/100079 A1; PCT Publication No. WO 2014/022758 A1; PCT Publication No. WO 2014/055897 A2; PCT Publication No. WO 2015/061668 A1; PCT Publication No. WO 2015/109124 A1; PCT Publication No. WO 2015/195163 A1; PCT Publication No. WO 2016/000619 A1; and PCT Publication No. WO 2016/030350 A1.

In certain embodiments, the human PD-1 inhibitor is pidilizumab.

### Methods of Treating Colorectal Cancer

The instant disclosure provides methods (in particular, neoadjuvant methods) for the treatment of colorectal cancer using a human CTLA-4 inhibitor (e.g., an antibody that specifically binds to human CTLA-4) and a human PD-1 inhibitor, and demonstrates, unexpectedly, that such methods can be used to treat non-MSI-H/dMMR colorectal cancer.

In one aspect, provided herein is a method of treating a colorectal tumor in a subject in need thereof, the method comprising administering to the subject: a dose of a first antibody that specifically binds to human CTLA-4; and a first dose of a human PD-1 inhibitor, wherein the dose of the first antibody and the first dose of the human PD-1 inhibitor are each administered to the subject prior to surgical removal of the tumor. In certain embodiments, the method further comprises administering to the subject: a second dose of the human PD-1 inhibitor, wherein the second dose is administered to the subject prior to surgical removal of the tumor. In certain embodiments, the colorectal cancer is not microsatellite instable—high (MSI-H). In certain embodiments, the colorectal cancer is microsatellite instable—high (MSI-H). In certain embodiments, the colorectal cancer is microsatellite stable (MSS). In certain embodiments, the colorectal cancer is not mismatch repair deficient (dMMR). In certain embodiments, the colorectal cancer is mismatch repair deficient (dMMR). In certain embodiments, the colorectal cancer is mismatch repair proficient (pMMR).

In another aspect, provided herein is a method of treating a colorectal tumor in a subject in need thereof, the method comprising administering to the subject: a dose of a first antibody that specifically binds to human CTLA-4; a first dose of a human PD-1 inhibitor; and a second dose of the human PD-1 inhibitor, wherein the dose of the first antibody and the first and second doses of the human PD-1 inhibitor are each administered to the subject prior to surgical removal of the tumor. In certain embodiments, the subject does not receive a chemotherapeutic agent as part of the neoadjuvant treatment. In certain embodiments, the colorectal cancer is not microsatellite instable—high (MSI-H). In certain embodiments, the colorectal cancer is microsatellite instable—high (MSI-H). In certain embodiments, the colorectal cancer is microsatellite stable (MSS). In certain embodiments, the colorectal cancer is not mismatch repair deficient (dMMR). In certain embodiments, the colorectal cancer is mismatch repair deficient (dMMR). In certain embodiments, the colorectal cancer is mismatch repair proficient (pMMR).

In certain embodiments, the first antibody is an antibody that specifically binds to human CTLA-4, wherein the first antibody comprises a human IgG heavy chain constant region that is a variant of a wild type human IgG heavy chain constant region, wherein the variant human IgG heavy chain constant region binds to FcγRIIIA with a higher affinity as compared to the affinity that the wild type human IgG heavy chain constant region binds to FcγRIIIA.

In certain embodiments, the first antibody is an antibody that specifically binds to human CTLA-4 and comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8. In certain embodiments, the first antibody comprises any of the anti-CTLA-4 antibodies described herein.

In certain embodiments, the human PD-1 inhibitor is a second antibody that specifically binds to human PD-1. In certain embodiments, the second antibody comprises: a VH comprising the CDRH1, CDRH2, and CDRH3 amino acid sequences of the VH amino acid sequence set forth in SEQ ID NO: 17; and a VL comprising the CDRL1, CDRL2, and CDRL3 amino acid sequences of the VL amino acid sequence set forth in SEQ ID NO: 18. In certain embodiments, the human PD-1 inhibitor comprises any of the human PD-1 inhibitors (e.g., anti-PD-1 antibodies, anti-PD-L1 antibodies) described herein.

In certain embodiments, the dose of the first antibody and the first dose of the human PD-1 inhibitor are administered on the same day. In certain embodiments, the dose of the first antibody and the first dose of the human PD-1 inhibitor are administered simultaneously. In certain embodiments, the dose of the first antibody is administered prior to (e.g., 30 minutes before, 1 hour before, 2 hours before, 4 hours before, 8 hours before, 12 hours before, 16 hours before, 20 hours before, 1 day before, 1.5 days before, 2 days before, 3 days before, etc.) the first dose of the human PD-1 inhibitor. In certain embodiments, the dose of the first antibody is administered after (e.g., 30 minutes after, 1 hour after, 2 hours after, 4 hours after, 8 hours after, 12 hours after, 16 hours after, 20 hours after, 1 day after, 1.5 days after, 2 days after, 3 days after, etc.) the first dose of the human PD-1 inhibitor.

In certain embodiments, the second dose of the human PD-1 inhibitor is administered 5 to 30 days, 7 to 21 days, 9 to 19 days, or 12 to 19 days after the first dose of the human PD-1 inhibitor is administered. In certain embodiments, the second dose of the human PD-1 inhibitor is administered 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the first dose of the human PD-1 inhibitor is administered. In certain embodiments, the second dose of the human PD-1 inhibitor is administered 14 days after the first dose of the human PD-1 inhibitor is administered.

In certain embodiments, the dose of the first antibody is 1 mg to 1000 mg, 5 mg to 500 mg, 10 mg to 250 mg, 25 mg to 200 mg, 25 mg to 150 mg, 25 mg to 100 mg, 25 mg to 75 mg, 50 mg to 200 mg, 50 mg to 175 mg, 50 mg to 150 mg, 50 mg to 125 mg, 50 mg to 100 mg, 75 mg to 250 mg, 75 mg to 200 mg, 75 mg to 175 mg, or 75 mg to 150 mg.

In certain embodiments, the dose of the first antibody is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg. In certain embodiments, the dose of the first antibody is about 75 mg.

In certain embodiments, the dose of the first antibody is 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg. In certain embodiments, the dose of the first antibody is 75 mg.

In certain embodiments, the first dose and/or the second dose of the human PD-1 inhibitor is 1 mg to 1000 mg, 100 mg to 750 mg, 100 mg to 500 mg, 100 mg to 400 mg, 100 mg to 250 mg, 100 mg to 240 mg, 150 mg to 650 mg, 150 mg to 500 mg, 150 mg to 250 mg, 150 mg to 240 mg, 200 mg to 750 mg, 200 mg to 650 mg, 200 mg to 500 mg, 200 mg to 300 mg, 200 mg to 240 mg, 240 mg to 750 mg, 240 mg to 650 mg, 240 mg to 500 mg, 240 mg to 400 mg, or 240 mg to 300 mg.

In certain embodiments, the first dose and/or the second dose of the human PD-1 inhibitor is about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 450 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, or about 740 mg. In certain embodiments, the first dose and/or the second dose of the human PD-1 inhibitor is about 240 mg. In certain embodiments, the first dose and the second dose of the human PD-1 inhibitor is about 240 mg. In certain embodiments, the first dose and/or the second dose of the human PD-1 inhibitor is about 450 mg. In certain embodiments, the first dose and the second dose of the human PD-1 inhibitor is about 450 mg.

In certain embodiments, the first dose and/or the second dose of the human PD-1 inhibitor is 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, 400 mg, 420 mg, 440 mg, 450 mg, 460 mg, 480 mg, 500 mg, 520 mg, 540 mg, 560 mg, 580 mg, 600 mg, 620 mg, 640 mg, 660 mg, 680 mg, 700 mg, 720 mg, or 740 mg. In certain embodiments, the first dose and/or the second dose of the human PD-1 inhibitor is 240 mg. In certain embodiments, the first dose and the second dose of the human PD-1 inhibitor is 240 mg. In certain embodiments, the first dose and/or the second dose of the human PD-1 inhibitor is 450 mg. In certain embodiments, the first dose and the second dose of the human PD-1 inhibitor is 450 mg.

In certain embodiments, the first dose and the second dose of the human PD-1 inhibitor are the same. In certain embodiments, the first dose and the second dose of the human PD-1 inhibitor are different.

In certain embodiments, the dose of the first antibody or the first dose of the human PD-1 inhibitor is administered 5 to 100 days, 7 to 98 days, 7 to 91 days, 7 to 84 days, 7 to 77 days, 7 to 70 days, 7 to 63 days, 7 to 56 days, 10 to 70 days, 12 to 67 days, 14 to 63 days, 14 to 56 days, 14 to 55 days, 14 to 45 days, 14 to 35 days, 14 to 30 days, 16 to 65 days, 16 to 61 days, 16 to 55 days, 16 to 45 days, 16 to 35 days, 16 to 30 days, 19 to 65 days, 19 to 61 days, 19 to 55 days, 19 to 50 days, 19 to 45 days, 19 to 40 days, 19 to 35 days, 19 to 30 days, 19 to 28 days, 21 to 70 days, 21 to 63 days, or 21 to 56 days before surgical removal of the tumor. In certain embodiments, the dose of the first antibody or the first dose of the human PD-1 inhibitor is administered 10 to 70 days, 14 to 63 days, 16 to 61 days, 19 to 61 days, or 21 to 56 days before surgical removal of the tumor. In certain embodiments, the dose of the first antibody or the first dose of the human PD-1 inhibitor is administered 19 to 61 days before surgical removal of the tumor.

In certain embodiments, the dose of the first antibody and the first dose of the human PD-1 inhibitor is administered 5 to 100 days, 7 to 98 days, 7 to 91 days, 7 to 84 days, 7 to 77 days, 7 to 70 days, 7 to 63 days, 7 to 56 days, 10 to 70 days, 12 to 67 days, 14 to 63 days, 14 to 56 days, 14 to 55 days, 14 to 45 days, 14 to 35 days, 14 to 30 days, 16 to 65 days, 16 to 61 days, 16 to 55 days, 16 to 45 days, 16 to 35 days, 16 to 30 days, 19 to 65 days, 19 to 61 days, 19 to 55 days, 19 to 50 days, 19 to 45 days, 19 to 40 days, 19 to 35 days, 19 to 30 days, 19 to 28 days, 21 to 70 days, 21 to 63 days, or 21 to 56 days before surgical removal of the tumor. In certain embodiments, the dose of the first antibody and the first dose of the human PD-1 inhibitor is administered 10 to 70 days, 14 to 63 days, 16 to 61 days, 19 to 61 days, or 21 to 56 days before surgical removal of the tumor. In certain embodiments, the dose of the first antibody and the first dose of the human PD-1 inhibitor is administered 19 to 61 days before surgical removal of the tumor.

In certain embodiments, the dose of the first antibody or the first dose of the human PD-1 inhibitor is administered 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days before surgical removal of the tumor. In certain embodiments, the dose of the first antibody or the first dose of the human PD-1 inhibitor is administered 21 days before surgical removal of the tumor. In certain embodiments, the dose of the first antibody or the first dose of the human PD-1 inhibitor is administered 28 days before surgical removal of the tumor. In certain embodiments, the dose of the first antibody or the first dose of the human PD-1 inhibitor is administered 35 days before surgical removal of the tumor. In certain embodiments, the dose of the first antibody or the first dose of the human PD-1 inhibitor is administered 42 days before surgical removal of the tumor. In certain embodiments, the dose of the first antibody or the first dose of the human PD-1 inhibitor is administered 49 days before surgical removal of the tumor. In certain embodiments, the dose of the first antibody or the first dose of the human PD-1 inhibitor is administered 56 days before surgical removal of the tumor. In certain embodiments, the dose of the first antibody or the first dose of the human PD-1 inhibitor is administered 63 days before surgical removal of the tumor. In certain embodiments, the dose of the first antibody or the first dose of the human PD-1 inhibitor is administered 70 days before surgical removal of the tumor. In certain embodiments, the dose of the first antibody or the first dose of the human PD-1 inhibitor is administered 77 days before surgical removal of the tumor. In certain embodiments, the dose of the first antibody or the first dose of the human PD-1 inhibitor is administered 84 days before surgical removal of the tumor. In certain embodiments, the dose of the first antibody or the first dose of the human PD-1 inhibitor is administered 91 days before surgical removal of the tumor. In certain embodiments, the dose of the first antibody or the first dose of the human PD-1 inhibitor is administered 98 days before surgical removal of the tumor.

In certain embodiments, the dose of the first antibody and the first dose of the human PD-1 inhibitor is administered 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days before surgical removal of the tumor. In certain embodiments, the dose of the first antibody and the first dose of the human PD-1 inhibitor is administered 21 days before surgical removal of the tumor. In certain embodiments, the dose of the first antibody and the first dose of the human PD-1 inhibitor is administered 28 days before surgical removal of the tumor. In certain embodiments, the dose of the first antibody and the first dose of the human PD-1 inhibitor is administered 35 days before surgical removal of the tumor. In certain embodiments, the dose of the first antibody and the first dose of the human PD-1 inhibitor is administered 42 days before surgical removal of the tumor. In certain embodiments, the dose of the first antibody and the first dose of the human PD-1 inhibitor is administered 49 days before surgical removal of the tumor. In certain embodiments, the dose of the first antibody and the first dose of the human PD-1 inhibitor is administered 56 days before surgical removal of the tumor. In certain embodiments, the dose of the first antibody and the first dose of the human PD-1 inhibitor is administered 63 days before surgical removal of the tumor. In certain embodiments, the dose of the first antibody and the first dose of the human PD-1 inhibitor is administered 70 days before surgical removal of the tumor. In certain embodiments, the dose of the first antibody or the first dose of the human PD-1 inhibitor is administered 77 days before surgical removal of the tumor. In certain embodiments, the dose of the first antibody or the first dose of the human PD-1 inhibitor is administered 84 days before surgical removal of the tumor. In certain embodiments, the dose of the first antibody or the first dose of the human PD-1 inhibitor is administered 91 days before surgical removal of the tumor. In certain embodiments, the dose of the first antibody or the first dose of the human PD-1 inhibitor is administered 98 days before surgical removal of the tumor.

In certain embodiments, the second dose of the human PD-1 inhibitor is administered at least 1 day, at least 2 days, at least 3 days, 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, at least 51 days, at least 52 days, at least 53 days, at least 54 days, at least 55 days, or at least 56 days before surgical removal of the tumor. In certain embodiments, the second dose of the human PD-1 inhibitor is administered at least 7 days before surgical removal of the tumor. In certain embodiments, the second dose of the human PD-1 inhibitor is administered at least 14 days before surgical removal of the tumor. In certain embodiments, the second dose of the human PD-1 inhibitor is administered at least 21 days before surgical removal of the tumor. In certain embodiments, the second dose of the human PD-1 inhibitor is administered at least 28 days before surgical removal of the tumor. In certain embodiments, the second dose of the human PD-1 inhibitor is administered at least 35 days before surgical removal of the tumor. In certain embodiments, the second dose of the human PD-1 inhibitor is administered at least 42 days before surgical removal of the tumor. In certain embodiments, the second dose of the human PD-1 inhibitor is administered at least 49 days before surgical removal of the tumor. In certain embodiments, the second dose of the human PD-1 inhibitor is administered at least 56 days before surgical removal of the tumor.

In certain embodiments, the second dose of the human PD-1 inhibitor is administered 1 to 100 days, 1 to 98 days, 1 to 91 days, 1 to 84 days, 1 to 77 days, 1 to 70 days, 1 to 63 days, 1 to 60 days, 5 to 45 days, 5 to 42 days, 5 to 40 days, 5 to 35 days, 5 to 30 days, 5 to 28 days, 5 to 25 days, 5 to 21 days, 5 to 18 days, 5 to 14 days, 7 to 98 days, 7 to 91 days, 7 to 84 days, 7 to 77 days, 7 to 70 days, 7 to 63 days, 7 to 56 days, 7 to 49 days, 7 to 45 days, 7 to 42 days, 7 to 35 days, 7 to 30 days, 7 to 28 days, 7 to 25 days, 7 to 21 days, 7 to 18 days, or 7 to 14 days before surgical removal of the tumor. In certain embodiments, the second dose of the human PD-1 inhibitor is administered 1 to 100 days, 1 to 60 days, 1 to 56 days, 5 to 45 days, 7 to 63 days, 7 to 56 days, 7 to 49 days, or 7 to 42 days before surgical removal of the tumor.

In certain embodiments, the second dose of the human PD-1 inhibitor is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days before surgical removal of the tumor. In certain embodiments, the second dose of the human PD-1 inhibitor is administered 7 days before surgical removal of the tumor. In certain embodiments, the second dose of the human PD-1 inhibitor is administered 14 days before surgical removal of the tumor. In certain embodiments, the second dose of the human PD-1 inhibitor is administered 21 days before surgical removal of the tumor. In certain embodiments, the second dose of the human PD-1 inhibitor is administered 28 days before surgical removal of the tumor. In certain embodiments, the second dose of the human PD-1 inhibitor is administered 35 days before surgical removal of the tumor. In certain embodiments, the second dose of the human PD-1 inhibitor is administered 42 days before surgical removal of the tumor. In certain embodiments, the second dose of the human PD-1 inhibitor is administered 49 days before surgical removal of the tumor. In certain embodiments, the second dose of the human PD-1 inhibitor is administered 56 days before surgical removal of the tumor. In certain embodiments, the second dose of the human PD-1 inhibitor is administered 63 days before surgical removal of the tumor. In certain embodiments, the second dose of the human PD-1 inhibitor is administered 70 days before surgical removal of the tumor. In certain embodiments, the second dose of the human PD-1 inhibitor is administered 77 days before surgical removal of the tumor. In certain embodiments, the second dose of the human PD-1 inhibitor is administered 84 days before surgical removal of the tumor. In certain embodiments, the second dose of the human PD-1 inhibitor is administered 91 days before surgical removal of the tumor. In certain embodiments, the second dose of the human PD-1 inhibitor is administered 98 days before surgical removal of the tumor.

In certain embodiments, the method further comprises administering to the subject one or more additional doses of the first antibody that specifically binds to human CTLA-4.

In certain embodiments, the method further comprises administering to the subject one or more additional doses of the human PD-1 inhibitor, e.g., a third, a fourth, or a fifth dose of the human PD-1 inhibitor.

In another aspect, provided herein is a method of treating a colorectal tumor in a subject in need thereof, the method comprising administering to the subject: a dose of a first antibody that specifically binds to human CTLA-4; a first dose of a human PD-1 inhibitor; a second dose of the human PD-1 inhibitor; and a third dose of the human PD-1 inhibitor, wherein the dose of the first antibody and the first, second, and third doses of the human PD-1 inhibitor are each administered to the subject prior to surgical removal of the tumor. In certain embodiments, the colorectal cancer is not microsatellite instable—high (MSI-H). In certain embodiments, the colorectal cancer is microsatellite instable—high (MSI-H). In certain embodiments, the colorectal cancer is microsatellite stable (MSS). In certain embodiments, the colorectal cancer is not mismatch repair deficient (dMMR). In certain embodiments, the colorectal cancer is mismatch repair deficient (dMMR). In certain embodiments, the colorectal cancer is mismatch repair proficient (pMMR).

In another aspect, provided herein is a method of treating a colorectal tumor in a subject in need thereof, the method comprising administering to the subject: a dose of a first antibody that specifically binds to human CTLA-4; a first dose of a human PD-1 inhibitor; a second dose of the human PD-1 inhibitor; a third dose of the human PD-1 inhibitor; and a fourth dose of the human PD-1 inhibitor, wherein the dose of the first antibody and the first, second, third, and fourth doses of the human PD-1 inhibitor are each administered to the subject prior to surgical removal of the tumor. In certain embodiments, the colorectal cancer is not microsatellite instable—high (MSI-H). In certain embodiments, the colorectal cancer is microsatellite instable—high (MSI-H). In certain embodiments, the colorectal cancer is microsatellite stable (MSS). In certain embodiments, the colorectal cancer is not mismatch repair deficient (dMMR). In certain embodiments, the colorectal cancer is mismatch repair deficient (dMMR). In certain embodiments, the colorectal cancer is mismatch repair proficient (pMMR).

In certain embodiments, the subject does not receive a chemotherapeutic agent as part of the neoadjuvant treatment.

In certain embodiments, the dose of the first antibody and the first dose of the human PD-1 inhibitor are administered on the same day. In certain embodiments, the dose of the first antibody and the first dose of the human PD-1 inhibitor are administered simultaneously. In certain embodiments, the dose of the first antibody is administered prior to (e.g., 30 minutes before, 1 hour before, 2 hours before, 4 hours before, 8 hours before, 12 hours before, 16 hours before, 20 hours before, 1 day before, 1.5 days before, 2 days before, 3 days before, etc.) the first dose of the human PD-1 inhibitor. In certain embodiments, the dose of the first antibody is administered after (e.g., 30 minutes after, 1 hour after, 2 hours after, 4 hours after, 8 hours after, 12 hours after, 16 hours after, 20 hours after, 1 day after, 1.5 days after, 2 days after, 3 days after, etc.) the first dose of the human PD-1 inhibitor.

In certain embodiments, the second dose of the human PD-1 inhibitor is administered 5 to 30 days, 7 to 21 days, 9 to 19 days, or 12 to 19 days after the first dose of the human PD-1 inhibitor is administered. In certain embodiments, the second dose of the human PD-1 inhibitor is administered 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the first dose of the human PD-1 inhibitor is administered. In certain embodiments, the second dose of the human PD-1 inhibitor is administered 14 days after the first dose of the human PD-1 inhibitor is administered.

In certain embodiments, the third dose of the human PD-1 inhibitor is administered 5 to 30 days, 7 to 21 days, 9 to 19 days, or 12 to 19 days after the second dose of the human PD-1 inhibitor is administered. In certain embodiments, the third dose of the human PD-1 inhibitor is administered 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the second dose of the human PD-1 inhibitor is administered. In certain embodiments, the third dose of the human PD-1 inhibitor is administered 14 days after the second dose of the human PD-1 inhibitor is administered.

In certain embodiments, the fourth dose of the human PD-1 inhibitor is administered 5 to 30 days, 7 to 21 days, 9 to 19 days, or 12 to 19 days after the third dose of the human PD-1 inhibitor is administered. In certain embodiments, the fourth dose of the human PD-1 inhibitor is administered 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the third dose of the human PD-1 inhibitor is administered. In certain embodiments, the fourth dose of the human PD-1 inhibitor is administered 14 days after the third dose of the human PD-1 inhibitor is administered.

In certain embodiments, the dose of the first antibody is 1 mg to 1000 mg, 5 mg to 500 mg, 10 mg to 250 mg, 25 mg to 200 mg, 25 mg to 150 mg, 25 mg to 100 mg, 25 mg to 75 mg, 50 mg to 200 mg, 50 mg to 175 mg, 50 mg to 150 mg, 50 mg to 125 mg, 50 mg to 100 mg, 75 mg to 250 mg, 75 mg to 200 mg, 75 mg to 175 mg, or 75 mg to 150 mg.

In certain embodiments, the dose of the first antibody is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg. In certain embodiments, the dose of the first antibody is about 75 mg.

In certain embodiments, the dose of the first antibody is 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg. In certain embodiments, the dose of the first antibody is 75 mg.

In certain embodiments, the first, second, third, and/or fourth dose of the human PD-1 inhibitor is 1 mg to 1000 mg, 100 mg to 750 mg, 100 mg to 500 mg, 100 mg to 400 mg, 100 mg to 250 mg, 100 mg to 240 mg, 150 mg to 650 mg, 150 mg to 500 mg, 150 mg to 250 mg, 150 mg to 240 mg, 200 mg to 750 mg, 200 mg to 650 mg, 200 mg to 500 mg, 200 mg to 300 mg, 200 mg to 240 mg, 240 mg to 750 mg, 240 mg to 650 mg, 240 mg to 500 mg, 240 mg to 400 mg, or 240 mg to 300 mg.

In certain embodiments, the first, second, third, and/or fourth dose of the human PD-1 inhibitor is about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 450 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, or about 740 mg. In certain embodiments, the first, second, third, and/or fourth dose of the human PD-1 inhibitor is about 240 mg. In certain embodiments, the first, second, third, and fourth dose of the human PD-1 inhibitor is about 240 mg. In certain embodiments, the first, second, third, and/or fourth dose of the human PD-1 inhibitor is about 450 mg. In certain embodiments, the first, second, third, and fourth dose of the human PD-1 inhibitor is about 450 mg.

In certain embodiments, the first, second, third, and/or fourth dose of the human PD-1 inhibitor is 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, 400 mg, 420 mg, 440 mg, 450 mg, 460 mg, 480 mg, 500 mg, 520 mg, 540 mg, 560 mg, 580 mg, 600 mg, 620 mg, 640 mg, 660 mg, 680 mg, 700 mg, 720 mg, or 740 mg. In certain embodiments, the first, second, third, and/or fourth dose of the human PD-1 inhibitor is 240 mg. In certain embodiments, the first, second, third, and fourth dose of the human PD-1 inhibitor is 240 mg. In certain embodiments, the first, second, third, and/or fourth dose of the human PD-1 inhibitor is 450 mg. In certain embodiments, the first, second, third, and fourth dose of the human PD-1 inhibitor is 450 mg.

In certain embodiments, the first, second, third, and fourth dose of the human PD-1 inhibitor are the same. In certain embodiments, the first, second, third, and fourth dose of the human PD-1 inhibitor are different.

In certain embodiments, the dose of the first antibody or the first dose of the human PD-1 inhibitor is administered 5 to 100 days, 7 to 98 days, 7 to 91 days, 7 to 84 days, 7 to 77 days, 7 to 70 days, 7 to 63 days, 7 to 56 days, 10 to 70 days, 12 to 67 days, 14 to 63 days, 14 to 56 days, 14 to 55 days, 14 to 45 days, 14 to 35 days, 14 to 30 days, 16 to 65 days, 16 to 61 days, 16 to 55 days, 16 to 45 days, 16 to 35 days, 16 to 30 days, 19 to 65 days, 19 to 61 days, 19 to 55 days, 19 to 50 days, 19 to 45 days, 19 to 40 days, 19 to 35 days, 19 to 30 days, 19 to 28 days, 21 to 70 days, 21 to 63 days, or 21 to 56 days before surgical removal of the tumor. In certain embodiments, the dose of the first antibody or the first dose of the human PD-1 inhibitor is administered 10 to 70 days, 14 to 63 days, 16 to 61 days, 19 to 61 days, or 21 to 56 days before surgical removal of the tumor. In certain embodiments, the dose of the first antibody or the first dose of the human PD-1 inhibitor is administered 19 to 61 days before surgical removal of the tumor.

In certain embodiments, the dose of the first antibody and the first dose of the human PD-1 inhibitor is administered 5 to 100 days, 7 to 98 days, 7 to 91 days, 7 to 84 days, 7 to 77 days, 7 to 70 days, 7 to 63 days, 7 to 56 days, 10 to 70 days, 12 to 67 days, 14 to 63 days, 14 to 56 days, 14 to 55 days, 14 to 45 days, 14 to 35 days, 14 to 30 days, 16 to 65 days, 16 to 61 days, 16 to 55 days, 16 to 45 days, 16 to 35 days, 16 to 30 days, 19 to 65 days, 19 to 61 days, 19 to 55 days, 19 to 50 days, 19 to 45 days, 19 to 40 days, 19 to 35 days, 19 to 30 days, 19 to 28 days, 21 to 70 days, 21 to 63 days, or 21 to 56 days before surgical removal of the tumor. In certain embodiments, the dose of the first antibody and the first dose of the human PD-1 inhibitor is administered 10 to 70 days, 14 to 63 days, 16 to 61 days, 19 to 61 days, or 21 to 56 days before surgical removal of the tumor. In certain embodiments, the dose of the first antibody and the first dose of the human PD-1 inhibitor is administered 19 to 61 days before surgical removal of the tumor.

In certain embodiments, the dose of the first antibody or the first dose of the human PD-1 inhibitor is administered 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days before surgical removal of the tumor. In certain embodiments, the dose of the first antibody or the first dose of the human PD-1 inhibitor is administered 21 days before surgical removal of the tumor. In certain embodiments, the dose of the first antibody or the first dose of the human PD-1 inhibitor is administered 28 days before surgical removal of the tumor. In certain embodiments, the dose of the first antibody or the first dose of the human PD-1 inhibitor is administered 35 days before surgical removal of the tumor. In certain embodiments, the dose of the first antibody or the first dose of the human PD-1 inhibitor is administered 42 days before surgical removal of the tumor. In certain embodiments, the dose of the first antibody or the first dose of the human PD-1 inhibitor is administered 49 days before surgical removal of the tumor. In certain embodiments, the dose of the first antibody or the first dose of the human PD-1 inhibitor is administered 56 days before surgical removal of the tumor. In certain embodiments, the dose of the first antibody or the first dose of the human PD-1 inhibitor is administered 63 days before surgical removal of the tumor. In certain embodiments, the dose of the first antibody or the first dose of the human PD-1 inhibitor is administered 70 days before surgical removal of the tumor. In certain embodiments, the dose of the first antibody or the first dose of the human PD-1 inhibitor is administered 77 days before surgical removal of the tumor. In certain embodiments, the dose of the first antibody or the first dose of the human PD-1 inhibitor is administered 84 days before surgical removal of the tumor. In certain embodiments, the dose of the first antibody or the first dose of the human PD-1 inhibitor is administered 91 days before surgical removal of the tumor. In certain embodiments, the dose of the first antibody or the first dose of the human PD-1 inhibitor is administered 98 days before surgical removal of the tumor.

In certain embodiments, the dose of the first antibody and the first dose of the human PD-1 inhibitor is administered 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days before surgical removal of the tumor. In certain embodiments, the dose of the first antibody and the first dose of the human PD-1 inhibitor is administered 21 days before surgical removal of the tumor. In certain embodiments, the dose of the first antibody and the first dose of the human PD-1 inhibitor is administered 28 days before surgical removal of the tumor. In certain embodiments, the dose of the first antibody and the first dose of the human PD-1 inhibitor is administered 35 days before surgical removal of the tumor. In certain embodiments, the dose of the first antibody and the first dose of the human PD-1 inhibitor is administered 42 days before surgical removal of the tumor. In certain embodiments, the dose of the first antibody and the first dose of the human PD-1 inhibitor is administered 49 days before surgical removal of the tumor. In certain embodiments, the dose of the first antibody and the first dose of the human PD-1 inhibitor is administered 56 days before surgical removal of the tumor. In certain embodiments, the dose of the first antibody and the first dose of the human PD-1 inhibitor is administered 63 days before surgical removal of the tumor. In certain embodiments, the dose of the first antibody and the first dose of the human PD-1 inhibitor is administered 70 days before surgical removal of the tumor. In certain embodiments, the dose of the first antibody or the first dose of the human PD-1 inhibitor is administered 77 days before surgical removal of the tumor. In certain embodiments, the dose of the first antibody or the first dose of the human PD-1 inhibitor is administered 84 days before surgical removal of the tumor. In certain embodiments, the dose of the first antibody or the first dose of the human PD-1 inhibitor is administered 91 days before surgical removal of the tumor. In certain embodiments, the dose of the first antibody or the first dose of the human PD-1 inhibitor is administered 98 days before surgical removal of the tumor.

In certain embodiments, the third dose of the human PD-1 inhibitor is administered at least 1 day, at least 2 days, at least 3 days, 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, at least 51 days, at least 52 days, at least 53 days, at least 54 days, at least 55 days, or at least 56 days before surgical removal of the tumor. In certain embodiments, the third dose of the human PD-1 inhibitor is administered at least 7 days before surgical removal of the tumor. In certain embodiments, the third dose of the human PD-1 inhibitor is administered at least 14 days before surgical removal of the tumor. In certain embodiments, the third dose of the human PD-1 inhibitor is administered at least 21 days before surgical removal of the tumor. In certain embodiments, the third dose of the human PD-1 inhibitor is administered at least 28 days before surgical removal of the tumor. In certain embodiments, the third dose of the human PD-1 inhibitor is administered at least 35 days before surgical removal of the tumor. In certain embodiments, the third dose of the human PD-1 inhibitor is administered at least 42 days before surgical removal of the tumor. In certain embodiments, the third dose of the human PD-1 inhibitor is administered at least 49 days before surgical removal of the tumor. In certain embodiments, the third dose of the human PD-1 inhibitor is administered at least 56 days before surgical removal of the tumor.

In certain embodiments, the third dose of the human PD-1 inhibitor is administered 1 to 100 days, 1 to 98 days, 1 to 91 days, 1 to 84 days, 1 to 77 days, 1 to 70 days, 1 to 63 days, 1 to 60 days, 5 to 45 days, 5 to 42 days, 5 to 40 days, 5 to 35 days, 5 to 30 days, 5 to 28 days, 5 to 25 days, 5 to 21 days, 5 to 18 days, 5 to 14 days, 7 to 98 days, 7 to 91 days, 7 to 84 days, 7 to 77 days, 7 to 70 days, 7 to 63 days, 7 to 56 days, 7 to 49 days, 7 to 45 days, 7 to 42 days, 7 to 35 days, 7 to 30 days, 7 to 28 days, 7 to 25 days, 7 to 21 days, 7 to 18 days, or 7 to 14 days before surgical removal of the tumor. In certain embodiments, the third dose of the human PD-1 inhibitor is administered 1 to 100 days, 1 to 60 days, 1 to 56 days, 5 to 45 days, 7 to 63 days, 7 to 56 days, 7 to 49 days, or 7 to 42 days before surgical removal of the tumor.

In certain embodiments, the third dose of the human PD-1 inhibitor is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days before surgical removal of the tumor. In certain embodiments, the third dose of the human PD-1 inhibitor is administered 7 days before surgical removal of the tumor. In certain embodiments, the third dose of the human PD-1 inhibitor is administered 14 days before surgical removal of the tumor. In certain embodiments, the third dose of the human PD-1 inhibitor is administered 21 days before surgical removal of the tumor. In certain embodiments, the third dose of the human PD-1 inhibitor is administered 28 days before surgical removal of the tumor. In certain embodiments, the third dose of the human PD-1 inhibitor is administered 35 days before surgical removal of the tumor. In certain embodiments, the third dose of the human PD-1 inhibitor is administered 42 days before surgical removal of the tumor. In certain embodiments, the third dose of the human PD-1 inhibitor is administered 49 days before surgical removal of the tumor. In certain embodiments, the third dose of the human PD-1 inhibitor is administered 56 days before surgical removal of the tumor. In certain embodiments, the third dose of the human PD-1 inhibitor is administered 63 days before surgical removal of the tumor. In certain embodiments, the third dose of the human PD-1 inhibitor is administered 70 days before surgical removal of the tumor. In certain embodiments, the third dose of the human PD-1 inhibitor is administered 77 days before surgical removal of the tumor. In certain embodiments, the third dose of the human PD-1 inhibitor is administered 84 days before surgical removal of the tumor. In certain embodiments, the third dose of the human PD-1 inhibitor is administered 91 days before surgical removal of the tumor. In certain embodiments, the third dose of the human PD-1 inhibitor is administered 98 days before surgical removal of the tumor.

In certain embodiments, the fourth dose of the human PD-1 inhibitor is administered at least 1 day, at least 2 days, at least 3 days, 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, at least 51 days, at least 52 days, at least 53 days, at least 54 days, at least 55 days, or at least 56 days before surgical removal of the tumor. In certain embodiments, the fourth dose of the human PD-1 inhibitor is administered at least 7 days before surgical removal of the tumor. In certain embodiments, the fourth dose of the human PD-1 inhibitor is administered at least 14 days before surgical removal of the tumor. In certain embodiments, the fourth dose of the human PD-1 inhibitor is administered at least 21 days before surgical removal of the tumor. In certain embodiments, the fourth dose of the human PD-1 inhibitor is administered at least 28 days before surgical removal of the tumor. In certain embodiments, the fourth dose of the human PD-1 inhibitor is administered at least 35 days before surgical removal of the tumor. In certain embodiments, the fourth dose of the human PD-1 inhibitor is administered at least 42 days before surgical removal of the tumor. In certain embodiments, the fourth dose of the human PD-1 inhibitor is administered at least 49 days before surgical removal of the tumor. In certain embodiments, the fourth dose of the human PD-1 inhibitor is administered at least 56 days before surgical removal of the tumor.

In certain embodiments, the fourth dose of the human PD-1 inhibitor is administered 1 to 100 days, 1 to 98 days, 1 to 91 days, 1 to 84 days, 1 to 77 days, 1 to 70 days, 1 to 63 days, 1 to 60 days, 5 to 45 days, 5 to 42 days, 5 to 40 days, 5 to 35 days, 5 to 30 days, 5 to 28 days, 5 to 25 days, 5 to 21 days, 5 to 18 days, 5 to 14 days, 7 to 98 days, 7 to 91 days, 7 to 84 days, 7 to 77 days, 7 to 70 days, 7 to 63 days, 7 to 56 days, 7 to 49 days, 7 to 45 days, 7 to 42 days, 7 to 35 days, 7 to 30 days, 7 to 28 days, 7 to 25 days, 7 to 21 days, 7 to 18 days, or 7 to 14 days before surgical removal of the tumor. In certain embodiments, the fourth dose of the human PD-1 inhibitor is administered 1 to 100 days, 1 to 60 days, 1 to 56 days, 5 to 45 days, 7 to 63 days, 7 to 56 days, 7 to 49 days, or 7 to 42 days before surgical removal of the tumor.

In certain embodiments, the fourth dose of the human PD-1 inhibitor is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days before surgical removal of the tumor. In certain embodiments, the fourth dose of the human PD-1 inhibitor is administered 7 days before surgical removal of the tumor. In certain embodiments, the fourth dose of the human PD-1 inhibitor is administered 14 days before surgical removal of the tumor. In certain embodiments, the fourth dose of the human PD-1 inhibitor is administered 21 days before surgical removal of the tumor. In certain embodiments, the fourth dose of the human PD-1 inhibitor is administered 28 days before surgical removal of the tumor. In certain embodiments, the fourth dose of the human PD-1 inhibitor is administered 35 days before surgical removal of the tumor. In certain embodiments, the fourth dose of the human PD-1 inhibitor is administered 42 days before surgical removal of the tumor. In certain embodiments, the fourth dose of the human PD-1 inhibitor is administered 49 days before surgical removal of the tumor. In certain embodiments, the fourth dose of the human PD-1 inhibitor is administered 56 days before surgical removal of the tumor. In certain embodiments, the fourth dose of the human PD-1 inhibitor is administered 63 days before surgical removal of the tumor. In certain embodiments, the fourth dose of the human PD-1 inhibitor is administered 70 days before surgical removal of the tumor. In certain embodiments, the fourth dose of the human PD-1 inhibitor is administered 77 days before surgical removal of the tumor. In certain embodiments, the fourth dose of the human PD-1 inhibitor is administered 84 days before surgical removal of the tumor. In certain embodiments, the fourth dose of the human PD-1 inhibitor is administered 91 days before surgical removal of the tumor. In certain embodiments, the fourth dose of the human PD-1 inhibitor is administered 98 days before surgical removal of the tumor.

In certain embodiments of any of the methods of treating colorectal cancer provided herein, the colorectal tumor is colorectal adenocarcinoma. In certain embodiments, the colorectal tumor is not metastatic. In certain embodiments, the colorectal tumor is a primary tumor. In certain embodiments, the subject has a RAS mutation. In certain embodiments, the RAS mutation is a KRAS or NRAS mutation.

In certain embodiments of any of the methods of treating colorectal cancer provided herein, the colorectal cancer is not microsatellite instable—high (MSI-H). In certain embodiments, the colorectal cancer is microsatellite instable—high (MSI-H). In certain embodiments, the colorectal cancer is microsatellite instable—low (MSI-L). In certain embodiments, the colorectal cancer is microsatellite stable (MSS). In certain embodiments, the colorectal cancer is not mismatch repair deficient (dMMR). In certain embodiments, the colorectal cancer is mismatch repair proficient (pMMR).

In certain embodiments, the subject has not received any prior chemotherapy. In certain embodiments, the subject has not received any prior radiation therapy.

In certain embodiments, the cancer may be positive or negative for expression of PD-L1 (e.g., when PD-L1 stained tumor infiltrating immune cells cover less than 5% of the tumor area as determined by immunohistochemistry or as determined by any commercially available companion diagnostic, including diagnostics with FDA premarket approval numbers P160002, P150013, or P150025).

In certain embodiments, the first antibody or the human PD-1 inhibitor is administered intravenously. In certain embodiments, the first antibody or the human PD-1 inhibitor is administered intratumorally. In certain embodiments, the first antibody or the human PD-1 inhibitor is administered peritumorally.

In certain embodiments, the first antibody and the human PD-1 inhibitor is administered intravenously. In certain embodiments, the first antibody and the human PD-1 inhibitor is administered intratumorally. In certain embodiments, the first antibody and the human PD-1 inhibitor is administered peritumorally.

In certain embodiments, the first antibody or the human PD-1 inhibitor is administered by intravenous infusion over about 30 minutes. In certain embodiments, the first antibody or the human PD-1 inhibitor is administered by intravenous infusion over about 45 minutes. In certain embodiments, the first antibody or the human PD-1 inhibitor is administered by intravenous infusion over about 60 minutes. In certain embodiments, the first antibody or the human PD-1 inhibitor is administered by intravenous infusion over about 90 minutes.

In certain embodiments, the first antibody and the human PD-1 inhibitor is administered by intravenous infusion over about 30 minutes. In certain embodiments, the first antibody and the human PD-1 inhibitor is administered by intravenous infusion over about 45 minutes. In certain embodiments, the first antibody and the human PD-1 inhibitor is administered by intravenous infusion over about 60 minutes. In certain embodiments, the first antibody and the human PD-1 inhibitor is administered by intravenous infusion over about 90 minutes.

In certain embodiments, the dose of the first antibody, the first dose of the human PD-1 inhibitor, or the second dose of the human PD-1 inhibitor is a therapeutically effective amount. In certain embodiments, the dose of the first antibody, the first dose of the human PD-1 inhibitor, and the second dose of the human PD-1 inhibitor is a therapeutically effective amount.

In certain embodiments, the subject is at least 18 years of age. In certain embodiments, the subject has histologically, cytologically, or clinically confirmed adenocarcinoma of the colon. In certain embodiments, before administration of the first antibody and/or the human PD-1 inhibitor, the subject has an Eastern Cooperative Oncology Group performance status of 0-2. In certain embodiments, before administration of the first antibody and/or the human PD-1 inhibitor, the subject has adequate organ and bone marrow reserve function as defined by one or more of: absolute neutrophil count≥1.5×10⁹ per L;
platelets≥100×10⁹ per L; hemoglobin≥8.0 g/dL without a transfusion that has occurred within 2 weeks of hemoglobin measurement; creatinine clearance≥40 mL/min as measured or calculated per local institutional standards; aspartate aminotransferase≤2.5× upper limit of normal (ULN); alanine aminotransferase≤2.5×ULN; and total bilirubin≤1.5×ULN.

In certain embodiments, before administration of the first antibody and/or the human PD-1 inhibitor, the subject does not have metastases identified using standard of care radiographic imaging. In certain embodiments, before administration of the first antibody and/or the human PD-1 inhibitor, the subject is not pregnant and/or is not breastfeeding. In certain embodiments, the subject has not received a live vaccination within 28 days prior to administration of the first antibody and/or the human PD-1 inhibitor. In certain embodiments, before administration of the first antibody and/or the human PD-1 inhibitor, the subject does not have clinically significant cardiovascular disease or an active infection requiring treatment. In certain embodiments, the subject has not received systemic corticosteroid therapy within 7 days prior to administration of the first antibody and/or the human PD-1 inhibitor.

In certain embodiments, administration of the first antibody and the human PD-1 inhibitor reduces residual viable tumor cells in the subject following surgical removal of the tumor. In certain embodiments, administration of the first antibody and the human PD-1 inhibitor reduces minimal residual disease in the subject following surgical removal of the tumor. In certain embodiments, minimal residual disease is assessed via measurement/detection of circulating tumor DNA.

In certain embodiments, administration of the first antibody and the human PD-1 inhibitor reduces the size of the tumor in the subject. In certain embodiments, administration of the first antibody and the human PD-1 inhibitor increases T-cell, memory T-cell, myeloid cell, and/or antigen presenting cell activation in the subject. In certain embodiments, administration of the first antibody and the human PD-1 inhibitor reduces the number of Treg cells in the subject.

In certain embodiments, administration of the first antibody and the human PD-1 inhibitor reduces the need for additional treatment (e.g., adjuvant chemotherapy) following surgical removal of the tumor.

In certain embodiments, administration of the first antibody and the human PD-1 inhibitor increases the level of one or more cytokine(s). In certain embodiments, the one or more cytokine is CXCL9 or CXCL10. In certain embodiments, administration of the first antibody and the human PD-1 inhibitor increases the level of IFN-γ, HLA-DR, and/or ICOS expression.

In certain embodiments, the methods herein result in about a 1, 5, 10, 20, 30, 40, 50, 60, 70, 80, 90, or 100% reduction in tumor burden in the subject. In certain embodiments, the methods herein result in a reduced tumor burden. In certain embodiments, the methods herein result in increased survival. In certain embodiments, the methods herein result in an increase in overall survival. In certain embodiments, the methods herein result in an increase in progression-free survival.

In an aspect, provided herein is an antibody that specifically binds to human CTLA-4 and a human PD-1 inhibitor for use in the treatment of a colorectal tumor, wherein the treatment is performed according to a method described herein.

In an aspect, provided herein is an antibody that specifically binds to human CTLA-4 and a human PD-1 inhibitor for use in the manufacture of a medicament for the treatment of a colorectal tumor, wherein the treatment is performed according to a method described herein.

In an aspect, provided herein is an antibody that specifically binds to human CTLA-4 and a human PD-1 inhibitor for the treatment of a colorectal tumor, wherein the treatment is performed according to a method described herein.

### EXAMPLES

### Example 1. Exploratory Study to Test Combination of Botensilimab and Balstilimab

Immunotherapy in Patients with Resectable Colorectal Cancer

This Example describes a single-center, single-arm, non-randomized, open-label, phase II, pilot study that was initiated to assess the feasibility, safety, and efficacy of using a combination of the CTLA-4 inhibitor botensilimab (“bot”) and the PD-1 inhibitor balstilimab (“bal”) in the neoadjuvant setting in patients with colorectal cancer, prior to resection. Patients with colorectal cancer received 2 or 4 doses of bal (each dose 2 weeks apart) and a single dose of bot within 6 weeks prior to resection. The combination of 1 dose of bot and 2 doses of bal is referred to in the Examples as the “bot/bal regimen.”

### Subject Selection

Subjects with a diagnosis of resectable, localized/advanced adenocarcinoma of the colon who met the inclusion and exclusion criteria were eligible for participation in this study.

In order to participate in the study, subjects met all of the following inclusion criteria:
1. capable of and willing to provide informed, written consent;
2. age greater than or equal to 18 years;
3. histologically, cytologically, or clinically confirmed adenocarcinoma of the colon;
4. plans for surgical resection as determined by the treating physician and principal investigator;
5. Eastern Cooperative Oncology Group (ECOG) performance status of less than or equal to 2;
6. adequate organ and bone marrow reserve function, as indicated by the following laboratory values:
   a. adequate hematological function, defined as absolute neutrophil count (ANC)≥1.5×10⁹/L, platelet count≥100×10⁹/L, and hemoglobin levels of ≥8 g/dL without recent transfusion (defined as a transfusion that has occurred within 2 weeks of the hemoglobin measurement);
   b. adequate liver function, defined as total bilirubin level≤1.5×upper limit of normal (ULN), aspartate aminotransferase (AST)≤2.5×ULN, and alanine aminotransferase (ALT)≤2.5×ULN; and
   c. adequate renal function defined as calculated creatinine clearance≥40 mL/min as determined by the Cockcroft-Gault equation; and
7. if capable of becoming pregnant, or getting someone else pregnant, must have been willing to use highly effective contraception from Screening period through 90 days following the last dose of study drug.

Subjects were excluded from the trial if any of the following criteria were met:
1. presence of metastases on routine, standard of care radiographic imaging (no stage 4 allowed);
2. previous treatment with immune checkpoint inhibitors targeting CTLA-4, PD-1 or PD-L1;
3. currently participating and receiving study therapy or has participated in a study of an investigational agent and received study therapy or used an investigation device within 3 weeks of first dose of current study drug;
4. persistent toxicity of National Cancer Institute (NCI) Common Terminology Criteria for Adverse Events (CTCAE) version 5.0 Grade>1 severity that is related to prior therapy (note: Sensory neuropathy or alopecia of Grade≤2 are acceptable);
5. known severe (Grade≥3) hypersensitivity reactions to fully human monoclonal antibodies, antibody, or severe reaction to immuno-oncology agents, such as colitis or pneumonitis requiring treatment with steroids; or has a history of interstitial lung disease, any history of anaphylaxis, or uncontrolled asthma;
6. pregnant or breastfeeding—individuals capable of becoming pregnant must have a negative serum B-HCG test within 72 hours prior to receiving the first dose of study drug; women were considered of non-childbearing potential if they met any of the following criteria:
   a. ≥45 years of age and has not had menses for >1 year;
   b. amenorrheic for >2 years without a hysterectomy and/or oophorectomy and follicle stimulating hormone value in the postmenopausal range upon pretrial (Screening) evaluation;
   c. status was post-hysterectomy, -oophorectomy, or -tubal ligation;
7. active infection requiring treatment;
8. active HIV, Hepatitis B or C with uncontrolled disease, as determined by the treating investigator or principal investigator (PI; exception: a patient with non-active, controlled disease would have been allowed to participate in the study);
9. active or history of autoimmune disease that requires systemic treatment within 2 years of the start of study drug (i.e., with use of disease-modifying agents, corticosteroids, or immunosuppressive drugs); subjects with autoimmune conditions requiring hormone replacement therapy or topical treatments were eligible;
10. live vaccination within 28 days prior to receiving the first dose of immunotherapy;
11. receiving systemic corticosteroid therapy 1 week prior to the first dose of study drug or receiving any other form of systemic immunosuppressive medication; corticosteroid use as a premedication for IV contrast allergies/reactions was allowed; subjects who were receiving daily corticosteroid replacement therapy were also an exception to this rule; daily prednisone at doses of ≤7.5 mg or equivalent hydrocortisone dose are examples of permitted replacement therapy; use of inhaled or topical corticosteroids is permitted;
12. clinically significant (i.e., active) cardiovascular disease: cerebral vascular accident/stroke or myocardial infarction within 6 months of enrollment, unstable angina, congestive heart failure (New York Heart Association class≥II), or serious uncontrolled cardiac arrhythmia requiring medication that may prevent surgery;
13. history or current evidence of any condition, therapy, any active infections, or laboratory abnormality that might confound the results of the trial, interfere with the subject's participation for the full duration of the trial, or is not in the best interest of the subject to participate, in the opinion of the treating Investigator or the PI.

### Study Procedures

Primary endpoints of the study included assessment of anti-tumor efficacy, safety, and feasibility of the bot/bal regimen. Anti-tumor effects of the treatments were assessed using pathological overall response (pOR) rate determined by analysis of tissue resected during surgery. Safety was assessed by evaluation of adverse events (AEs) and serious AEs according to the Common Terminology Criteria for Adverse Events (CTCAE) v5.0 at 90 days following the last treatment with balstilimab or botensilimab. Feasibility was assessed by evaluating complications leading to delays of 12 weeks or more in surgery after initiation of treatment (Day 0). Changes in minimal residual disease was assessed using ctDNA pre- and post-surgical resection.

The correlatives for this study were broadly divided into immune correlates (pre- and post-immunotherapy) and tumor correlates (pre- and post-immunotherapy and surgery on the surgical specimen). The specifics and timing of sample collection are described below. Both in-house research/clinical as well as commercially available platforms were utilized to accomplish the listed goals.

A main goal was to study the main correlates (tissue/blood pre- and post-immunotherapy) from a) an immune perspective and b) a tumor perspective. As noted in detail below, additional serial blood testing collections were taken to allow for assessment of blood-based immune markers as well as tumor based liquid biopsies (e.g., circulating tumor DNA testing).

Analysis of immune cells by flow cytometry: Analysis of immune cell populations via flow cytometry allowed for more quantitative and in-depth profiling of immune cells.

Immune cell analysis of peripheral blood and tumor tissue: Frequencies of innate (monocytes, macrophages, neutrophils, dendritic cells, NK cells) and adaptive (CD4+ T-cells, CD8+ T-cells, NKT cells, B cells) immune cells present in the peripheral blood or tumors of patients were determined via flow cytometry. In addition, expression of inhibitory markers on T-cells, including, but not limited to, PD-1, CTLA-4, TIM3 and LAG3 were examined. For peripheral blood samples, frequencies of immune cell populations were compared at the several serial time points described below. Tumor infiltrating cells in tumor tissue samples were compared at two time points for the tissue as described below.

Multi-plex immunofluorescence (IF) and scanning: FFPE pre-treatment biopsy and post-treatment surgical resection colorectal samples were mounted on glass slides for multiplexed IF analysis using the Orion™ system (RareCyte, Inc.). Briefly, FFPE sections (5 μm) on glass slides were baked, dewaxed with xylene, antigen-retrieved (BioGenex EZ-Retriever with EZ-AR 2 Elegance buffer), quenched to reduce endogenous tissue fluorescence, then stained manually with the RareCyte 13-plex Immuno-oncology panel for the following biomarkers: Nucleus (Hoechst), CD3, CD4, CD8, CD20, CD31, CD68, CD163, FOXP3, Ki67, PanCK, PD-1, PD-L1 (Table 3). The stained slides were imaged on the Orion instrument, and the resulting scan files were processed to TIFF images for downstream quantitative analysis. Hematoxylin and eosin staining and scanning of the FFPE slides were also performed. Briefly, sections were baked, dewaxed with xylene, then stained manually with hematoxylin and eosin Y. The stained slides were imaged on a CyteFinder HT instrument and the resulting scan files were processed to TIFF images for downstream analysis.

TABLE 3
Multiplex IF panel markers.

| Analyte | Definition |
|---|---|
| CD20+ cell density | CD20+ count (primarily B cells) per mm² of tumor area |
| CD68+ cell density | CD68+ count (primarily Macrophages) per mm² of tumor area |
| CD163+ cell density | CD163+ count (primarily Macrophages) per mm² of tumor area |
| CD3+ cell density | CD3+ count (primarily T cells) per mm² of tumor area |
| CD4+ cell density | CD4+ count (T helper cells [Th] and some macrophages) per mm² of |
| | tumor area |
| CD8+ cell density | CD8+ count (primarily cytotoxic T lymphocytes [CTL]) per mm² of |
| | tumor area |
| FOXP3+ cell density | FOXP3+ count (primarily regulatory T cells [Treg]) per mm² of tumor |
| | area |
| FOXP3+ fraction | FOXP3+ as a percentage of Th cells (CD3+ cells minus CD8+ cells) |
| PD-1+ cell density | PD-1+ count (primarily a subset of T cells) per mm² of tumor area |
| PD-L1 CPS score | Combined Positive Score: PD-L1+ immune and tumor cells as a |
| | percentage of total cells (tumor, immune, stromal) |
| PD-L1 TPS score | Total Proportional Score: PD-L1+ tumor cells as a percentage of total |
| | tumor cells |
| Immune Proliferation | Ki67+ immune cells as a percentage of total immune cells |
| Index | |
| Tumor Hot/Cold | Extent and inflammatory status of T-cell tumor infiltration; high (Hot) |
| | or low (Cold) |

Quantitative image analysis: Pre- and post-treatment cell densities within Tumor ROI(s) were measured using a quantitative image analysis pipeline to determine the impact of treatment on the immune response in the tumor microenvironment. In brief, each image was segmented into cells which were then classified into populations using MFI-based cutoffs that were set manually for each slide. Tumor ROI(s) were identified by pathologist review of H&E and IF images to determine spatial regions for cell population density measurements. Cell densities were calculated by summing the number of each cell type across all tumor ROIs and dividing by the total ROI area. Quantitative image analysis results were then reviewed by a pathologist to confirm trends in cell density. Changes in immune cell content after treatment within the tumor were calculated by taking the ratio of each immune cell density after and before treatment. Deviations to analysis were completed when required when pre-treatment samples were unavailable, and/or when no viable tumor was present in the resection. When pre-treatment samples were unavailable, the pathologist identified normal mucosa ROIs within the post-treatment resection and compared the resulting cell densities between the tumor and normal ROIs. When no viable tumor remained, the pathologist identified regions of necrotic tumor and drew ROIs around the immune response surrounding the necrotic tumor. Results were compiled and presented with descriptive statistics summarizing the intra- and inter-patient results and trends/patterns observed.

Serum cytokine and chemokine analysis: Use of a Multiplex assay allowed for examination of multiple cytokine and chemokine targets at multiple time points with use of minimal serum sample. These analyses were performed on-site at the time of sample collection. Serum was collected from peripheral blood of patients at the different time points as described below. A bead based multiplex assay was used to quantitatively measure levels of different cytokines and chemokines at various time points.

From a tumor microenvironment perspective, immune related changes including but not limited to PD-L1 and PD-1 expression and tumor-infiltrating lymphocytes were evaluated.

Tumor correlates: Tissue tumor evaluation was performed at two timepoints, i.e., pre- and post-immunotherapy (on the standard of care biopsy sample at diagnosis if possible, and on the surgical specimen post therapy).

Additionally, liquid biopsies (for ctDNA/circulating tumor DNA testing) were performed at multiple serial timepoints. The variant allele fraction (%) and the spectrum of mutations were measured via next generation sequencing based platforms. Tumor mutation burden was estimated and studied. On tissue sequencing was used to assess mutational status and guideline recommended markers. If insufficient tissue was available from the biopsy sample, analysis was reserved primarily for the surgical specimen.

Radiological staging: For patients with rectal cancer, MRI is routinely performed both for TNM staging and restaging and for identifying status of the circumferential resection margin, extramural venous invasion, and tumor deposits and aiding in surgical planning, especially for patients with low rectal cancers (Fernandes et al., 2022, Surg. Oncol. 43:101739). Local staging of colon cancer is not routinely performed with imaging, likely due to differences in systemic and surgical treatment for colon cancer versus rectal cancer and the relatively poor performance of CT for local staging (Nerad et al., 2016, AJR Am. J. Roentgenol. 207:984-995). However, it is important to note that prior studies have shown high sensitivity and low specificity of CT for detection of T3/T4 disease and low sensitivity and specificity for detection of nodal involvement. MRI is not widely used for staging of colon cancer, despite relatively high sensitivity and specificity for detecting T3/T4 tumors and moderate sensitivity and specificity for detection of nodal involvement (Nerad et al., 2017, Dis. Colon Rectum 60:385-392). A dedicated radiologist blinded to the results assessed the staging in patients with an MRI for rectal cancer and/or CT for colon cancer.

A summary schedule of treatments and assessments performed prior to surgical resection is shown in Table 4. A summary schedule of post-surgery follow-up assessments is shown in Table 5. A participant is considered to have completed the study if he or she had completed all phases of the study including the last visit or the last scheduled procedure as described in Table 5. Further description of the visits, treatments, and assessments is included below.

TABLE 4
Schedule of pre-surgery assessments.

| | | | | Toxicity | Toxicity |
|---|---|---|---|---|---|
| | | | Treatment | Check | Check |
| | | Treat- | Visit 2 | pre-op 1^{D} | pre-op 2^{D} |
| | Screening | ment | Day 14 | Day 28 | Day 42 |
| | −28 to −1 | Visit 1 | (−2 to +5 | (+/−7 | (+/−7 |
| | Days | Day 0 | days) | days) | days) |
| Balstilimab | | X | X | | |
| administration | | | | | |
| Botensilimab | | X | | | |
| administration | | | | | |
| Informed | X | | | | |
| consent | | | | | |
| Demographics | X | | | | |
| Medical | X | | | | |
| history | | | | | |
| Concurrent | X | X | X | | |
| medications | | | | | |
| Complete | X | X | X | | |
| physical exam | | | | | |
| Vital signs | X | X | X | | |
| Height | X | | | | |
| Weight | X | X | X | | |
| β-HCG | X^{C} | | | | |
| CBC w/ | X | X | X | | |
| differential | | | | | |
| and platelets | | | | | |
| Serum | X | X | X | | |
| chemistry^{A} | | | | | |
| EKG | X | | | | |
| (as indicated) | | | | | |
| Adverse event | | X | X | | |
| evaluation | | | | | |
| Tumor tissue | X | | | | |
| collection^{B} | | | | | |
| Biomarker | | X | X | X | X |
| blood draw/ | | | | | |
| Research | | | | | |

^{A}Albumin, alkaline phosphatase, total bilirubin, bicarbonate, BUN, calcium, chloride, creatinine, glucose, LDH, phosphorus, potassium, total protein, SGOT[AST], SGPT[ALT], sodium.
^{B}Please note tumor tissue if not present was not an exclusion. But if archival was present, correlative studies would be done on both the biopsy/surgery sample. Similarly, collection of tumor tissue was not required in the off-study period, but if obtained as part of standard of care, was used for correlative studies.
^{C}Serum pregnancy test (people capable of becoming pregnant).
^{D}Toxicity bloodwork continued every 2 weeks (±7 days) until surgery; if surgery was done prior to Day 28 or Day 42, those blood draws were not performed.

TABLE 5
Schedule of post-surgery assessments.

| | Post-op | Post-op | | |
|---|---|---|---|---|
| | Follow up | Follow up | | |
| | Visit 1 | Visit 2 | | |
| | 1 to 3 | 4 to 6 | Telephone | Off-Study^{D} |
| | weeks | weeks | Follow up | (Long-Term |
| | post-op | post-op | Day 104 | Follow Up) |
| | (+/−7 | (+/−7 | (+/−7 | Approx. |
| | days) | days) | days) | Q3 M-Q6 M |
| Concurrent meds | X | X | | X |
| Complete Physical | X | X | | X |
| exam | | | | |
| Vital signs | X | X | | X |
| Weight | X | X | | |
| CBC w/ | X | X | | X |
| differential and | | | | |
| platelets | | | | |
| Serum chemistry^{A} | X | X | | X |
| Adverse event | X | X | X | |
| evaluation | | | | |
| Tumor tissue | X^{C} | | | |
| collection^{B} | | | | |
| Biomarker blood | X | X | | |
| draw/ Research | | | | |
| Recurrence/ | | X | | X |
| Cancer Treatment | | | | |
| Data | | | | |
| Mortality | | X | | X |

^{A}Albumin, alkaline phosphatase, total bilirubin, bicarbonate, BUN, calcium, chloride, creatinine, glucose, LDH, phosphorus, potassium, total protein, SGOT[AST], SGPT[ALT], sodium.
^{B}Please note tumor tissue if not present was not an exclusion. But if archival was present, correlative studies would be done on both the biopsy/surgery sample. Similarly, collection of tumor tissue was not required in the off-study period, but if obtained as part of standard of care, was used for correlative studies.
^{C}Tumor tissue collection listed in follow-up visit 1 refers to tumor tissue that was made available to the research team from surgical resection.
^{D}Off-study evaluations were conducted per standard clinical practice; all evaluations were strongly encouraged to be completed, but not required if they were not consistent with standard clinical practice; data may be abstracted from the patient's medical records if care was not continued under study personnel; long-term follow up continued until occurrence of recurrence or death, whichever first.

### Screening Visit

A screening visit occurred within 28 days before start of treatment. The visit included: informed consent; demographics; medical history; concurrent medications; complete physical exam; vitals with height and weight; serum β-human chorionic gonadotropin (β-HCG), if applicable; complete blood count (CBC) with differential and platelets; serum chemistry (Complete Metabolic Panel [CMP]); electrocardiogram (EKG), as applicable; and tumor tissue collection, if available from previous standard of care procedure.

Standard of care, pre-operative imaging should be available to confirm that the patient does not have metastases, but this imaging was not required to be from within the 28-day screening period, as long as it is acceptable per standard clinical practice as determined by the treating physician or study principal investigator.

### Treatment Phase

During the treatment phase, all eligible subjects were assigned to three study cohorts. Patients in cohort A received 240 mg of balstilimab IV and 75 mg of botensilimab IV, followed by a second dose of 240 mg IV balstilimab 14 days (with a window of −2 to +5 days) after administration of the first dose. Patients in cohort B will receive four doses of balstilimab (240 mg IV each), administered approximately 2 weeks apart, and a single dose of botensilimab (75 mg IV) administered on the same day as the first dose of balstilimab. Patients in cohort C will receive the same regimen as patients in cohort B, but only includes patients with dMMR/MSI-H colorectal cancer. The cohort C patients will have the option of watch and wait. Additional planned cohorts include a cohort of 56 pMMR/MSS colon cancer patients and a cohort of 30 pMMR/MSS rectal cancer patients.

The first treatment visit (baseline; Day 1) included: Balstilimab administration; Botensilimab administration; concurrent medications; complete physical exam; vitals with weight; CBC with differential and platelets; serum chemistry (CMP); AE evaluation; biomarker/research blood draw.

The second treatment visit (Day 14; −2 to +5 days) included: Balstilimab administration; concurrent medications; complete physical exam; vitals with weight; CBC with differential and platelets; serum chemistry (CMP); AE evaluation; biomarker/research blood draw.

Patients in cohorts B and C will have third and fourth treatment visits to receive the third and fourth doses of balstilimab. The third treatment visit will be 14 days after the second treatment visit (−2 to +5 days), and the fourth treatment visit will be 14 days after the third treatment visit (−2 to +5 days).

Botensilimab and balstilimab were administered via continuous intravenous (IV) infusion over 30 (±5) minutes. No dose modifications were allowed for either drug. Participants that experienced dose limiting toxicities (DLTs) were permanently discontinued from study drug. The period of the DLT assessment began on Day 1 (first dose) for each subject and continued for 28 days post-surgery. A DLT was defined as the occurrence of any drug-related toxicity as detailed below that was clearly not associated with the underlying disease, a concomitant medication, or comorbidity. Toxicity was graded according to NCI CTCAE version 5.0.

General DLTs included any death not due to disease under investigation. Hematologic DLTs include: grade 3 thrombocytopenia with clinically significant bleeding (i.e., required hospitalization, transfusion of blood products, or other urgent medical intervention), or Grade 4 thrombocytopenia regardless of bleeding; grade≥3 febrile neutropenia (ANC<1.0×10⁹/L and fever>101° F./38.3° C.); grade 4 neutropenia (lasted more than 5 days); and grade 4 anemia, unless explained by underlying disease.

Non-hematologic DLTs included:
Any Grade≥3 non-hematologic AEs, whether immune-related or not, was considered a DLT with the exception of:
   Any Grade 3 endocrinopathy that was controlled with systemic corticosteroid therapy and/or hormone replacement therapy
   Grade 3 AE of tumor flare (defined as local pain, irritation, or rash localized at sites of known or suspected tumor)
   Grade 3 fatigue
   Transient (<72 hours) nausea, vomiting, and/or diarrhea in the absence of maximal medical therapy
   Grade≥3 electrolyte abnormality that lasted<72 hours, unless the patient had clinical symptoms
   Asymptomatic amylase and lipase elevation
Any Grade≥2 drug-related uveitis or eye pain or reduction of visual acuity was adjudicated as a DLT if it did not respond to topical therapy and did not improve to Grade 1 severity within 2 weeks of initiation of topical therapy OR if it required systemic treatment
Toxicities that indicated a high risk of a fatal drug-induced liver injury causing hepatocellular injury (not cholestatic injury)
Hy's Law: >3-fold elevations above the patient's baseline of alanine aminotransferase (ALT) or aspartate aminotransferase (AST), with elevation of serum total bilirubin to >2× patient's baseline, and without initial findings of cholestasis (elevated serum alkaline phosphatase [AP])

Dosing/procedure-related toxicities included any Grade>3 infusion reaction AE.

All concomitant medications were recorded and/or updated on subject medication log throughout the course of the study and saved in subject binder, if applicable.

In the absence of treatment delays due to adverse event(s), treatment continued for 2 (or 4) doses of balstilimab and 1 dose of botensilimab or until one of the following criteria applied: disease progression; intercurrent illness that prevented further administration of treatment; unacceptable adverse event(s); subject decided to withdraw from the study; or general or specific changes in the subject's condition render the subject unacceptable for further treatment in the judgment of the investigator.

A first toxicity check pre-op visit (Day 28; ±7 days) included biomarker/research blood draw. A second toxicity check pre-op visit (Day 42; ±7 days) included biomarker/research blood draw. Toxicity bloodwork continued every 2 weeks (±7 days) until surgery; if surgery was done prior to Day 28 or Day 42, those blood draws were not performed.

### Surgical Resection

Surgical resection was performed within 1-6 weeks after Treatment Visit 2 (for cohort A) or within 1-6 weeks after Treatment Visit 4 (for cohorts B and C). Surgery was performed per standard clinical practice by the treating physicians. No research specific procedures were performed. The research team had access to any laboratory testing performed as a part of standard pre-operative and intra-operative care via the participant's medical record. The research team received samples of the resected tumor tissue for research purposes, as well as standard pathology reports.

### Follow-up Phase

The follow-up phase began after completion of the surgical resection. Short-term follow-up was defined as the period from completion of surgery until completion of both the follow-up visit (1-6 weeks post-op) and the AE/SAE collection period (90 days following the last treatment with balstilimab or botensilimab). Once both the AE/SAE evaluation period and the follow-up visit was completed, the patient was considered off study and in long term follow-up. They were followed per clinical practice and no longer received any study-specific procedures. While every effort was made to complete all evaluations listed in the “Off-Study” column of Table 5, once the patient was considered off study, missing assessment and out of window assessments were no longer considered protocol deviations. Study personnel maintained access to the patient's NYP electronic medical record during the off-study period in order to collect data relevant to the study's exploratory endpoints, as well as information pertaining to oncological and treatment-related variables. All patients were followed off-study until recurrence or death, whichever occurred first. Participants removed from study for unacceptable adverse events were followed until resolution or stabilization of the adverse event.

Note: it was not required that any tumor tissue be collected during the follow-up phase, and no tissue was collected for research-only purposes, but if tissue was collected as part of standard of care, it was made available for correlative studies.

A first follow-up visit (Short-Term Follow-Up), 1 to 3 weeks post-op (+/−7 days), included: concurrent medications; complete physical exam; vitals with weight; CBC with differential and platelets; serum chemistry (CMP); AE evaluation; and biomarker/research blood draw.

A second follow-up visit (Short-Term Follow-Up), 4 to 6 weeks post-op (+/−7 days), included: concurrent medications; complete physical exam; vitals with weight; CBC with differential and platelets; serum chemistry (CMP); AE evaluation; biomarker/research blood draw; recurrence/cancer treatment data collection; and mortality data collection.

To ensure all reportable AEs/SAEs were recorded, participants were contacted via phone call at the end of the AE/SAE collection period and evaluated for AEs. Additionally, participants received continued medical care as part of their standard treatment and research staff reviewed participant medical records, as appropriate.

Off study evaluation (Long-Term Follow-Up) included, where possible: concurrent medications; complete physical exam; vitals; CBC with differential and platelets; serum chemistry (CMP); tumor tissue collection, if available from standard of care procedure; biomarker blood draw; recurrence/cancer treatment data collection; and mortality data collection.

### Stud Intervention Discontinuation and Participant Discontinuation Withdrawal

Discontinuation from botensilimab and balstilimab did not mean discontinuation from the study, and remaining study procedures were completed as indicated by the study protocol. If a clinically significant finding was identified (including, but not limited to changes from baseline) after enrollment, the investigator or qualified determined if any change in participant management was needed. Any new clinically relevant finding was reported as an adverse event (AE).

The data to be collected at the time of study intervention discontinuation included the following: evaluation of AEs and blood draw to assess CBC, CMP, ctDNA, and other biomarkers pertaining to immunological and tumor DNA in the tissue tumor/blood.

Participants were free to withdraw from participation in the study at any time upon request. Participants were discontinued or withdrawn from the study for the following reasons:
Pregnancy
Significant study intervention non-compliance
If any clinical adverse event (AE), laboratory abnormality, or other medical condition or situation occurred such that continued participation in the study would not be in the best interest of the participant
Disease progression which required discontinuation of the study intervention
If the participant met an exclusion criterion (either newly developed or not previously recognized) that precluded further study participation
Participant was unable to receive required dose of botensilimab or balstilimab within the timeframe defined in the schedule of assessments
Participant lost to follow-up after several attempts to contact participant to schedule study visit.

The reason for participant discontinuation or withdrawal from the study was recorded on the appropriate electronic Case Report Form (eCRF). Subjects who signed the informed consent form and are randomized but did not receive the study intervention could be replaced. Subjects who signed the informed consent form and received the study intervention, and subsequently withdrew, or were withdrawn or discontinued from the study, were not replaced.

A participant was considered lost to follow-up if he or she failed to return for any of the scheduled visits and was unable to be contacted by the study site staff.

The following actions were taken if a participant failed to return to the clinic for a required study visit:
The site attempted to contact the participant and rescheduled the missed visit within the timeframe designated in the schedule of assessments and counselled the participant on the importance of maintaining the assigned visit schedule and ascertained if the participant wished to and/or should continue in the study.
Before a participant was deemed lost to follow-up, the investigator or designee made every effort to regain contact with the participant (where possible, 3 telephone calls and, if necessary, a certified letter to the participant's last known mailing address or local equivalent methods). These contact attempts were documented in the participant's medical record or study file.
Should the participant continue to be unreachable, he or she was considered to have withdrawn from the study with a primary reason of lost to follow-up.

### Measurement of Effect

### Pathological Response Criteria

Resected tumors were examined in their entirety, and regression of resected tumors was assessed by estimating the percentage of residual viable tumor of the macroscopically identifiable tumor bed, as identified on routine hematoxylin and eosin (H&E) staining. In addition, regression was classified using the Mandard tumor regression grading system. Major pathologic response (MPR) was defined as ≤10% of residual viable tumor cells (or ≥90% response), corresponding to Mandard tumor regression grade 1 (CR) or 2 (near-CR). PR was defined as at least 50% tumor regression. Tumors with >50% and <90% residual viable tumor were labeled accordingly as ‘10-50% tumor regression,’ as per the NICHE study (Chalabi et al., 2020, Nat. Med. 26(4):566-576). When analyzing pMMR responders versus pMMR nonresponders, the subgroup was included in the group of nonresponders. Pathological response category definitions are shown in Table 6.

TABLE 6
Pathological response category definitions.

| Pathological Response Category | Definition |
|---|---|
| Major pathological response | ≤10% of residual viable tumor cells |
| (MPR; CR + near-CR) | (or ≥90% response) |
| 10-50% tumor regression | Tumors with >50% and <90% residual |
| | viable tumor |
| No response | ≤10% tumor regression |

ctDNA Assessment

Changes in circulating tumor DNA (ctDNA) were assessed using next generation sequencing/minimal residual disease (MRD)-based platforms before and after surgery. Changes in MRD were assessed. Summary statistics included mean, standard deviation, median, and range for ctDNA levels obtained at various time points (baseline, day 0, day 14, day 45, and day 60). Linear mixed-effects models were used to model longitudinal biomarker values. Simultaneous testing of general linear hypotheses were used to evaluate contrasts of interest.

### Adverse Events

The investigator was required to provide appropriate information concerning any findings that suggested significant hazards, contraindications, side effects, or precautions pertinent to the safe use of the drug or device under investigation. Safety was monitored by evaluation of adverse events reported by subjects or observed by investigators or research staff, as well as by other investigations such as clinical laboratory tests, x-rays, electrocardiographs, etc.

An adverse event (also referred to as an adverse experience) included any unfavorable and unintended sign (e.g., an abnormal laboratory finding), symptom, or disease temporally associated with the use of a drug and did not imply any judgment about causality. An adverse event could arise with any use of the drug (e.g., off-label use, use in combination with another drug) and with any route of administration, formulation, or dose, including an overdose.

Only pregnancies considered by the Investigator as related to study drug (e.g., resulting from a drug interaction with a contraceptive medication) were considered as AEs. Investigators actively followed up, documented, and reported the outcome of these pregnancies, even if subjects were withdrawn from the trial.

### Investigational Agent or Device Risks (Expected Adverse Events)

TABLE 7
SARs for Bal considered expected for safety reporting purposes.

| | | | Number of Patients Exposed | | |
|---|---|---|---|---|---|
| | | | (N = 595) | | |
| | | | | Life- | Fatal |
| System Organ | | Frequency | All SARs | Threatening | SARs |
| Class | Preferred Term | Category | n (%) | SARs n (%) | n (%) |
| Gastrointestinal | Immune-mediated | Common | 21 (3.5) | 1 (0.2) | 0 |
| disorders | enterocolitis^{A} | | | | |
| | Diarrhoea | Common | 11 (1.8) | 0 | 0 |
| | Colitis | Uncommon | 7 (1.2) | 0 | 0 |
| Respiratory, | Pneumonitis | Common | 5 (0.8) | 0 | 1 (0.2) |
| thoracic and | Immune-mediated | Uncommon | 2 (0.3) | 0 | 0 |
| mediastinal | lung disease^{B} | | | | |
| disorders | | | | | |
| Renal and | Immune-mediated | Uncommon | 3 (0.5) | 0 | 1 (0.2) |
| urinary | nephritis | | | | |
| disorders | Nephritis | Uncommon | 0 | 0 | 0 |
| Endocrine | Adrenal | Uncommon | 3 (0.5) | 0 | 0 |
| disorders | insufficiency | | | | |
| | Immune-mediated | Uncommon | 0 | 0 | 0 |
| | adrenal | | | | |
| | insufficiency | | | | |
| | Hypophysitis | Uncommon | 1 (0.2) | 0 | 0 |
| | Immune-mediated | Uncommon | 1 (0.2) | 0 | 0 |
| | hypophysitis | | | | |

Abbreviations: SAR: serious adverse reaction.
Events included diarrhoea, colitis, pneumonitis, nephritis, adrenal insufficiency, and hypophysitis was only considered expected if assessed as related to balstilimab or immune related.
All life-threatening and fatal events were considered unexpected for safety reporting.
Note:
N is the number of patients exposed to balstilimab in Agenus-sponsored studies (593) plus 2 patients from the Investigator-sponsored study who experienced a SAR; n is the number of patients who experienced a SAR.
^{A}Two events of immune-mediated enterocolitis were from the Investigator-sponsored study. Other data from this study were not included in this report.
^{B}Only events with lower-level term of immune-mediated pneumonitis would be considered expected.

Additionally, arthralgia is recognized as a risk of balstilimab. Utilizing a Standardized MedDRA Query (SMQ), 56 TEAEs were identified as having been assessed as related to balstilimab, including 45 events of arthralgia and 11 additional adverse drug reactions (ADRs) from SMQ Arthritis (3 ADRs of immune-mediated arthritis, 2 ADRs of arthritis, and 1 ADR each of polyarthritis, musculoskeletal stiffness, joint swelling, joint stiffness, joint range of motion decreased, and neck pain). All 56 ADRs were nonserious and mild to moderate in severity (42 Grade 1 events and 14 Grade 2 events).

Due to the potential of monoclonal antibodies to cause immune-mediated reactions, and as arthralgia is not a symptom of underlying malignancies, arthralgia was identified as a non-important, identified risk for balstilimab. A strategy for mitigation of this risk included symptomatic treatment and medical management for patients who developed symptoms following administration of balstilimab.

TABLE 8
SARs for Bot considered expected for safety reporting purposes.

| | | | Number of patients exposed | | |
|---|---|---|---|---|---|
| | | | (N) = 143 | | |
| | | | | | Life- |
| | | | | | Threat- |
| System | | | All | Fatal | ening |
| Organ | Preferred | Frequency | SARs | SARs | SARS |
| Class | Term^{A} | Category | n (%) | n (%) | n (%) |
| Metabolism | Dehydration^{B} | Common | 3 (2.1) | 0 | 0 |
| and | | | | | |
| Nutrition | | | | | |
| disorders | | | | | |
| Gastro- | Colitis | Common | 4 (2.8) | 0 | 0 |
| intestinal | Diarrhoea | Common | 6 (4.2) | 0 | 0 |
| disorders | Immune- | Common | 16 (11.2) | 1 (0.7) | 1 (0.7) |
| | mediated | | | | |
| | enterocolitis | | | | |

Abbreviations: IMP: investigational medicinal product; SAR: serious adverse reaction.
^{A}Lowest Level Terms within the preferred term were considered expected.
^{B}Dehydration was considered expected only if it was associated with another SAR.
Note:
n = number of patients who have experienced the SAR.

### Adverse Event Characteristics and Related Attributions

CTCAE term (AE description) and grade: The descriptions and grading scales found in the revised NCI Common Terminology Criteria for Adverse Events (CTCAE) version 5.0 were utilized for AE reporting (CTCAE version 5.0 was accessed from the CTEP web site (ctep.cancer.gov)).

Attribution of the AE:
Definite—The AE was clearly related to the study treatment.
Probable—The AE was likely related to the study treatment.
Possible—The AE could be related to the study treatment.
Unlikely—The AE was doubtfully related to the study treatment.
Unrelated—The AE was clearly NOT related to the study treatment.

### Recording of Adverse Events

All adverse events were captured from signing of the informed consent form through 90 days following the last treatment with balstilimab or botensilimab and recorded on a subject specific AE log. The AE log was maintained by the research staff and kept in the subject's research chart.

### Events of Special Interest

An AESI may be serious or nonserious. The rapid reporting of AESIs allows ongoing surveillance of these events in to characterize and understand them in association with the use of this investigational product.

The following types of AEs were considered AESIs:
IRRs
Hypersensitivity/anaphylactic reactions
irAEs, including but not limited to the following
Immune-mediated pneumonitis
Immune-mediated nephritis
Immune-mediated colitis
Immune-mediated hepatitis
Immune-mediated adrenal insufficiency
Abnormal hepatic function meeting Hy's Law Criteria

IRRs and hypersensitivity/anaphylactic reactions with a different underlying pharmacological etiology were considered AESIs. Anaphylaxis and IRRs have some common manifestations and may be difficult to distinguish from each other. IRRs are commonly observed during or shortly after the first time of exposure to therapeutic mAbs delivered through IV infusion. These reactions are less common following subsequent exposures. Unlike IRRs, anaphylaxis is a rare allergic mediated event, usually occurring after subsequent exposure to an antigen, and it is most commonly accompanied by severe systemic, skin and/or mucosal reactions. The investigator was advised to carefully examine adverse reactions observed during or shortly after drug infusion and considered the above-mentioned facts prior to making a final diagnosis.

AESIs for immune checkpoint inhibitors included, but were not limited to, events with a potential inflammatory or immune-mediated mechanism and which required more frequent monitoring and/or interventions such as steroids, immunosuppressants, and/or hormone replacement therapy. An irAE is defined as an AE that is associated with drug exposure and was consistent with an immune-related mechanism of action and where there was no clear alternate etiology. Serologic, immunologic, and histologic (biopsy) data, as appropriate, was used to support an irAE diagnosis. Appropriate efforts were made to rule out neoplastic, infectious, metabolic, toxin, or other etiologic causes of the irAE. Some potential irAEs included colitis, hepatitis, pneumonitis, nephritis, and adrenal insufficiency.

Cases where a patient demonstrated elevations in liver biochemistry could require further evaluation and may need to be reported as SAEs. The criteria for a potential Hy's Law case were AST or ALT≥3×ULN together with TBL≥2×ULN at any point during the study following the start of study medication irrespective of an increase in ALP.

### Serious AEs (SAEs)

SAEs included death, life threatening adverse experiences, hospitalization or prolongation of hospitalization, disability or incapacitation, overdose, congenital anomalies and any other important medical events that may jeopardize the subject or require medical or surgical intervention to prevent one of the outcomes listed in this definition.

### AE SAE Follow Up

All SAEs and AEs reported during this study were followed until resolution or until the investigator confirmed that the AE/SAE has stabilized, and no more follow-up is required. This requirement indicates that follow-up could be required for some events after the subject discontinues participation from the study. Any SAEs which were suspected to be related to product after the end of reporting period were sent to Agenus Inc.

The occurrence of an AE or SAE would come to the attention of study personnel during study visits and interviews of a study participant presented for medical care, via review of medical records, or upon review by a study monitor.

All AEs including local and systemic reactions not meeting the criteria for SAEs were captured on the appropriate eCRF. Information to be collected included event description, time of onset, clinician's assessment of severity, relationship to study product (assessed only by those with the training and authority to make a diagnosis), and time of resolution/stabilization of the event. All AEs that occurred while on study must be documented appropriately regardless of relationship. All AEs were followed to adequate resolution.

Any medical condition that was present at the time that the participant was screened was considered as baseline and not reported as an AE. If the study participant's condition deteriorated at any time during the study, it was recorded as an AE.

Changes in the severity of an AE were documented to allow an assessment of the duration of the event at each level of severity to be performed. AEs characterized as intermittent required documentation of onset and duration of each episode.

Research staff recorded all reportable events with start dates that occurred any time after informed consent was obtained until 90 days following the last treatment with balstilimab or botensilimab. At each study visit, the investigator inquired about the occurrence of AE/SAEs since the last visit. Events were followed for outcome information until resolution or stabilization.

### Results

### Patient Characteristics

Twelve cohort A patients were enrolled in the study as described above: 9 patients with pMMR/MSS CRC and 3 patients with dMMR/MSI-H colorectal cancer (CRC). Patient characteristics are summarized in Table 9, below. The median age was in the 50s (range 25-78 years). Equal number of females and males participated (n=6 of each; 50%). Patients belonged to a wide variety of races/backgrounds, and only a third (4/12; 33.3%) were Caucasian. In patients with dMMR/MSI-H CRC, 2 patients had germline Lynch Syndrome with germline MSH2 mutations. A spectrum of locations of the primary tumor were noted for patients enrolled in the study: 2 in the cecum, 1 in the ascending colon, 1 on the right side of the transverse colon, 1 in the center of the transverse colon, 3 on the left side of the sigmoid colon, and 4 in the sigmoid colon above the rectum. Thirteen cohort B patients and 1 cohort C patient were also enrolled in the study. Except where explicitly noted, the results described below are based on cohort A patients.

TABLE 9
Baseline cohort A patient characteristics
according to MSI status (n = 12).

| | |
|---|---|
| pMMR/MSS | dMMR/MSI-H tumors |
| tumors (n = 9) | (n = 3_{—} |

Age at enrollment (years)

| | | |
|---|---|---|
| Median (range) | 57.4 (25-78) | 51.6 (35-72) |

Sex (n(%))

| | | |
|---|---|---|
| Female | 5 (55%) | 1 (33%) |
| Male | 4 (45%) | 2 (67%) |

Race/Origin

| | | |
|---|---|---|
| Caucasian | 2 (22%) | 2 (67%) |
| African American | 3 (33%) | — |
| Hispanic | 1 (11%) | — |
| Middle Eastern/Arab | 1 (11%) | 1 (33%) |
| Southeast Asian | 2 (22%) | — |

Clinical disease stage

| | | |
|---|---|---|
| I | 1 (11%) | — |
| II | — | — |
| IIIA | 1 (11%) | — |
| IIIB | 4 (44%) | 1 (33%) |
| IIIC | 2 (22%) | 2 (66%) |
| Not classified | 1 (11%) | — |
| Lynch syndrome | 0 (0) | 2 (67%) |

### Safety and Feasibility

Neoadjuvant bot/bal was found to be safe and did not delay planned surgery in any patient. There were two instances of Grade 3 Treatment-Related Adverse Events (TRAEs). Only one patient (NEST-ID-11) had Grade 3 diarrhea/colitis which resolved on the same day after administration of a single dose of infliximab 10 mg/kg. This is the same patient who also had the pathologic complete response (100% tumor regression; 0% viable tumor remaining; see below). Five patients, of which four were females, experienced what is called “early immune activation syndrome”. This is characterized by fever (typically high-grade 101-102° F.), chills, and flu-like symptoms noted typically within 1-2 weeks of the bot/bal dose.

The supportive care plan was to be proactive about taking naproxen as a non-steroid anti-inflammatory drug (NSAID) or acetaminophen as an anti-pyretic, and in some instances prednisone 20 mg pills if necessary for 2-3 days if the fevers did not abate and/or were accompanied by systemic symptoms of feeling unwell. This usually resolved without additional interventions and patients got their planned PD-1 inhibitor (bal) as scheduled. In only one female patient who had fevers accompanied by grade-3 fatigue, the PD-1 inhibitor (bal) was delayed by a few days, since the patient did not pick up and take the NSAIDs or prednisone low dose as suggested in the protocol due to language barrier/communication issue. Once the NSAID and the low dose of prednisone were taken, the symptoms of early immune activation syndrome abated quickly to allow for completion of the planned neoadjuvant therapy and subsequent surgery.

### Efficacy and Pathologic Response

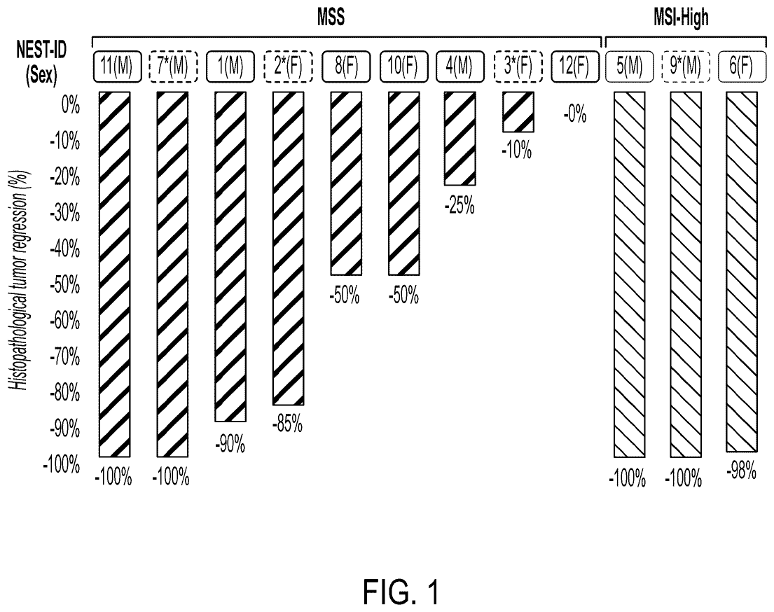

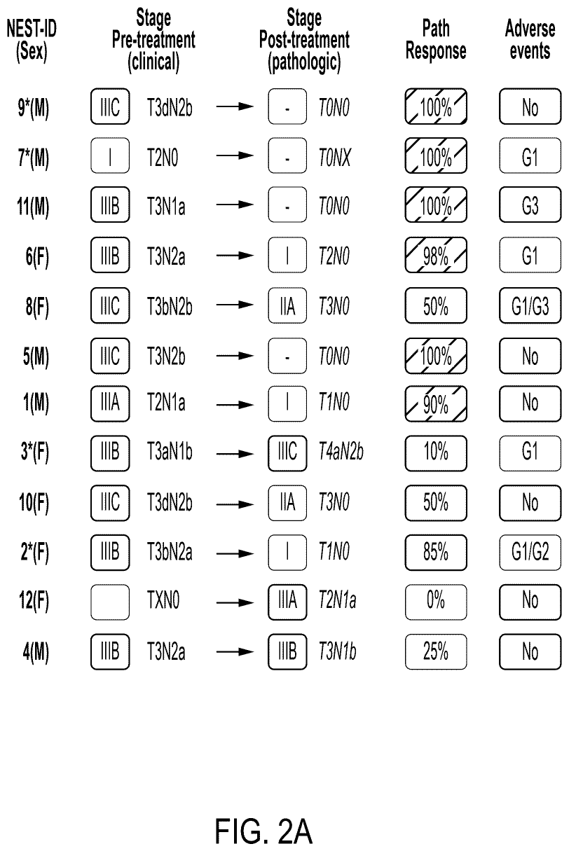

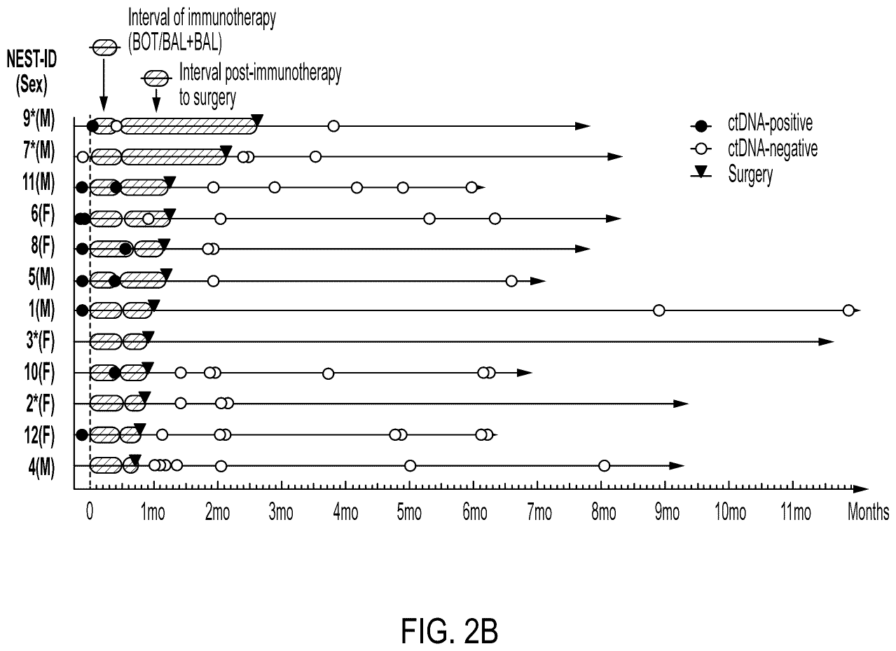

Measures of pathologic response (%) in each of the 12 cohort A patients enrolled in the trial is shown in FIG. 1. 6/9 (67%) patients with pMMR/MSS had a ≥50% pathologic response; 2/9 had pathologic complete responses. 3/3 (100%) of patients with dMMR/MSI-H had a major pathologic response (≥90% pathologic reduction). As of Mar. 18, 2024, with a median follow-up of 8 months (range 6-12 months), no cohort A patients had clinical, radiographic, or molecular recurrence (FIGS. 2A and 2B). 39 circulating tumor DNA (ctDNA)-minimal residual disease (MRD)-based assay timepoints were negative, showing sustained ctDNA clearance (FIGS. 2A and 2B). The downstaging noted is also outlined alongside the follow-up to date. CRC staging was assessed according to the American Joint Committee on Cancer: Chapter 20—Colon and Rectum, in: AJCC Cancer Staging Manual. 8th ed. New York, NY: Springer; 2017.

For assessment of the pathologic response, the resection specimens were thoroughly examined by pathology, with residual tumor and/or treatment sites being submitted entirely for histologic evaluation. Microscopically, pathologic complete response was identified in 4 patients (two patients with pMMR/MSS and two with dMMR/MSI-H tumors). The tumor beds showed a very dense, mixed inflammatory infiltrate, comprised largely of lymphocytes, plasma cells, and histiocytes, mucosal-based granulation tissue with neovascularization, and mucin pools. Eosinophils and neutrophils were also variably present. A Crohn-like reaction (tertiary lymphoid structures surrounding tumor bed) was exuberant in most cases. The infiltrates were associated with submucosal fibrosis and demonstrated striking necrotizing and non-necrotizing granulomatous inflammation with a lymphohistiocytic cuff. One patient's tumor (98% response—NEST-ID-6) showed residual viable tumor (2%) surrounded by extracellular mucin. Multiple layers of cells were observed. The outer layer consisted of a thick layer of predominantly T helper, T cytotoxic, CD163+/CD68-macrophages, and a few CD68+ macrophages. The inner layer consisted of a thin layer of the same cell types, but with a higher density of CD68+ macrophages, including CD68+ macrophages that were approaching or contacting the tumor cells. Between the outer and inner layer was mucin that was impeding cells from contacting the tumor cells. These above-mentioned tumors showed strong PDL1 positivity within the immune cells post-treatment.

In patients with ≥50% response, the inflammation was dense and like that seen above, most prominent at the periphery of viable tumor and deep within the wall of the colon. Residual tumor was often superficially oriented and within the center of the tumor bed, rather than the haphazard arrangement often seen in the setting of chemotherapy (“inside-out”-serosa-to-mucosa response; Kasi et al., 2023, Oncogene 42:3252-3259). Residual tumor glands frequently demonstrated evidence of ongoing destruction with disrupted crypts, incomplete lumens and frequent luminal microabscesses and necrosis. Regardless of response to therapy, increased expression of PD-L1 within inflammatory cells was noted in the surgical specimen post-treatment as compared to pre-treatment biopsies (Table 10).

TABLE 10
PD-L1 positive immune and tumor cells as a percentage
of total cells (tumor, immune, stromal).

| | | Post-treatment surgical |
|---|---|---|
| Patient | Pre-treatment biopsy | specimen |
| Patient 1 | 30 | 60 |
| Patient 2 | N/A | 5 |
| Patient 3 | <1 | 1 |
| Patient 4 | 1 | 1 |
| Patient 5 | 20 | 100 |
| Patient 6 | N/A | N/A |
| Patient 7 | N/A | 25 |
| Patient 8 | 3 | 100 |
| Patient 9 | N/A | 100 |
| Patient 10 | 0 | 10 |
| Patient 11 | 10 | 40 |
| Patient 12 | N/A | 70 |

In the dMMR/MSI-H group, striking necrotizing and non-necrotizing granulomatous inflammation was seen with lymphohistiocytic cells surrounding the tumor bed. These infiltrates were associated with submucosal fibrosis along with prominent neo-vascularization in the mucosal-based granulation tissue. In the pMMR/MSS group, pathological assessment of residual tumor revealed frequent disrupted crypts with neutrophilic microabscesses. Inflammation was dense and was prominently seen at the tumor periphery, deep within the colonic wall. The residual tumor was present superficially within the center of the tumor bed. Ongoing necrotic destruction was seen in the residual tumor glands with evidence of crypt destruction and incomplete lumens.

### Multiplex Immunofluorescence

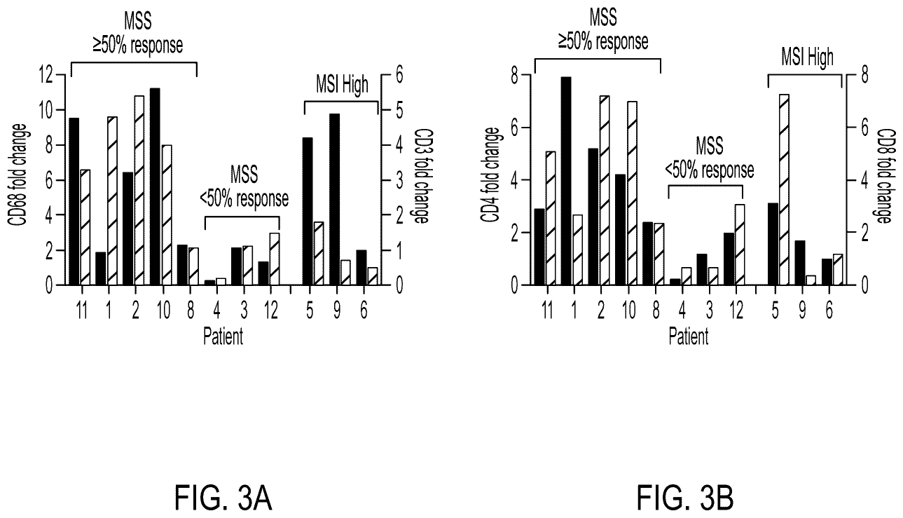

Based on the depth of response, patients with pMMR/MSS tumors were divided arbitrarily into those with ≥50% response (“high responders”—n=6 out of 9, 67%) versus those with <50% response (“moderate responders”—n=3 out of 9, 33%) as assessed by a pathologist. Similar to the histopathological findings, patients with ≥50% response were observed to have increased immune response compared to those with <50% response when measuring immune cell density in pre-treatment vs post-treatment samples (FIGS. 3A and 3B, Table 11). Median fold change in CD68 (6.4 vs 1.3), CD3 (4.0 vs 1.1), CD4 (4.2 vs 1.2) and CD8 (5.1 vs 0.7) were all elevated in exceptional versus moderate responders. Patient NEST-ID 8, a female with an exceptional pathologic response but having the smallest immune response, received steroids over the course of treatment for ‘early immune activation syndrome’ as noted earlier, that could have affected the final immune response assessment.

All patients with dMMR/MSI-H tumors had a major pathologic response (“exceptional responders”≥90% response; 98-100% pathologic response, n=3 out of 3, 100% with 2 out of 3 pathologic complete responses). Overall, an elevated immune response was observed as noted (FIGS. 3A and 3B, Table 11). Additionally, a 12-day post-treatment biopsy was analyzed for patient NEST-ID-9, which showed an elevated immune response compared to the post-treatment surgical resection. In particular, density of CD68 macrophages (1,088 vs 620 cells/mm²), T-cells (2,302 vs 1,000 cells/mm²), T-helper cells (2,025 vs 823 cells/mm²), T-cytotoxic cells (491 vs 134 cells/mm²) were all elevated in the 12-day post-treatment biopsy as compared to the surgical resection.

TABLE 11
Pathologic responses and changes in CD68, CD3, CD4, and CD8 ratios.

| | Category | | | | | | | | | | |
|---|---|---|---|---|---|---|---|---|---|---|---|
| | pMMR/MSS ≥50% | | | | | pMMR/MSS <50% | | | | | |
| | response | | | | | response | | | dMMR/MSI-H | | |
| | Patient ID (Sex) | | | | | | | | | | |
| | 11(M)^{‡} | 1(M) | 2(F)* | 10(F) | 8(F) | 4(M) | 3(F)^{†} | 12(F) | 5(M)^{‡} | 9(M)^{‡†} | 6(F)^{†} |
| Path | 100 | 90 | 85 | 50 | 50 | 25 | 10 | 0 | 100 | 100 | 98 |
| Response | | | | | | | | | | | |
| (%) | | | | | | | | | | | |
| Days to | 38 | 30 | 24 | 27 | 36 | 21 | 29 | 29 | 34 | 57 | 42 |
| surgery | | | | | | | | | | | |
| from | | | | | | | | | | | |
| cycle 1 | | | | | | | | | | | |
| day 1 | | | | | | | | | | | |
| CD68 | 9.5 | 1.9 | 6.4 | 11.2 | 2.3 | 0.21 | 2.1 | 1.3 | 8.4 | 9.8 | 2.0 |
| ratio | | | | | | | | | | | |
| CD3 ratio | 3.3 | 4.8 | 5.4 | 4.0 | 1.1 | 0.19 | 1.1 | 1.5 | 3.6 | 1.4 | 1.0 |
| CD4 ratio | 2.9 | 7.9 | 5.2 | 4.2 | 2.4 | 0.21 | 1.2 | 2.0 | 3.1 | 1.7 | 0.98 |
| CD8 ratio | 5.1 | 2.7 | 7.2 | 7.0 | 2.4 | 0.67 | 0.67 | 3.1 | 7.3 | 0.34 | 1.2 |
| Average | 5.2 | 4.3 | 6.1 | 6.6 | 2.1 | 0.3 | 1.3 | 2.0 | 5.6 | 3.3 | 1.3 |

Circulating Tumor DNA (ctDNA) Correlates

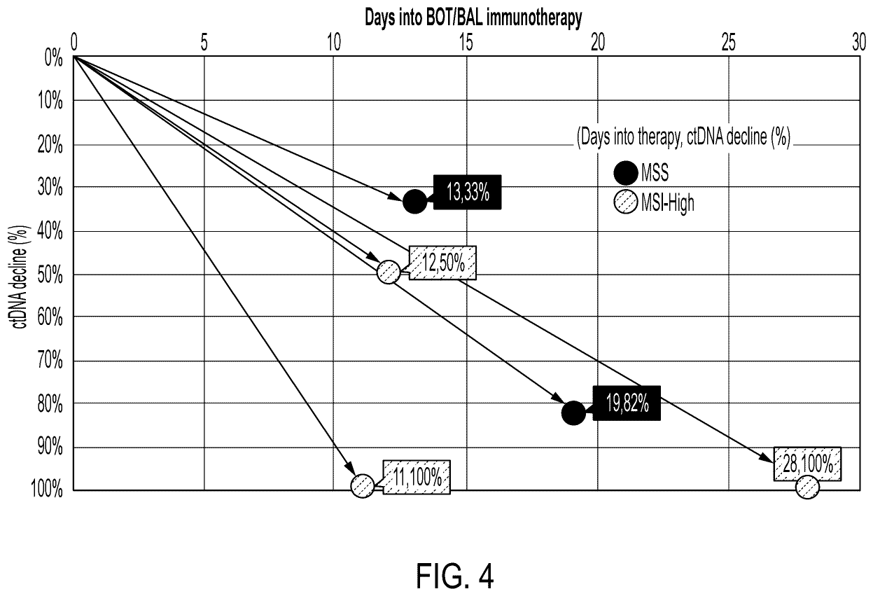

FIG. 4 summarizes the results of serial ctDNA testing. All patients who were positive for ctDNA at screening cleared ctDNA (7/7-100%) post-operatively. The decline in ctDNA in patients who had pre-operative testing on immunotherapy ranged from 33% to 100% (FIG. 4). Furthermore, 11/11 (100%) tested post-operatively have remained ctDNA/MRD negative to date on 39-MRD testing assay timepoints showing sustained ctDNA clearance.

### Tissue-Based Genetic Testing

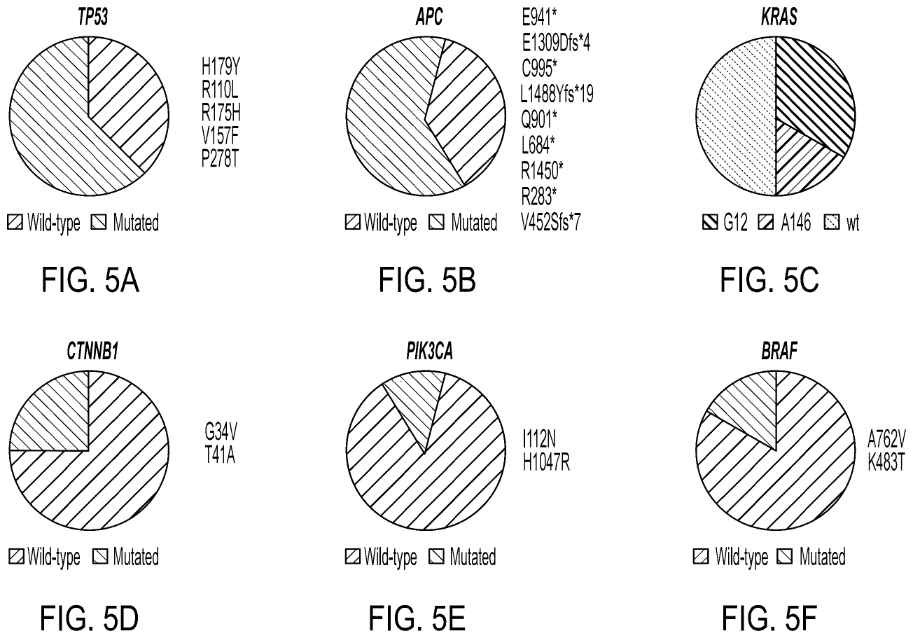

Similar to what is reported for patients with colorectal cancer, 6 of 12 (50%) of the patients were noted to be KRAS-mutant (FIG. 5C). FIGS. 5A-5F show the proportion of patients that had mutations in TP53 (FIG. 5A), APC (FIG. 5B), KRAS (FIG. 5C), PIK3CA (FIG. 5D), CTNNBI (FIG. 5E), and BRAF (FIG. 5F), with the specific mutations indicated.

### Circulatory Immune Correlates

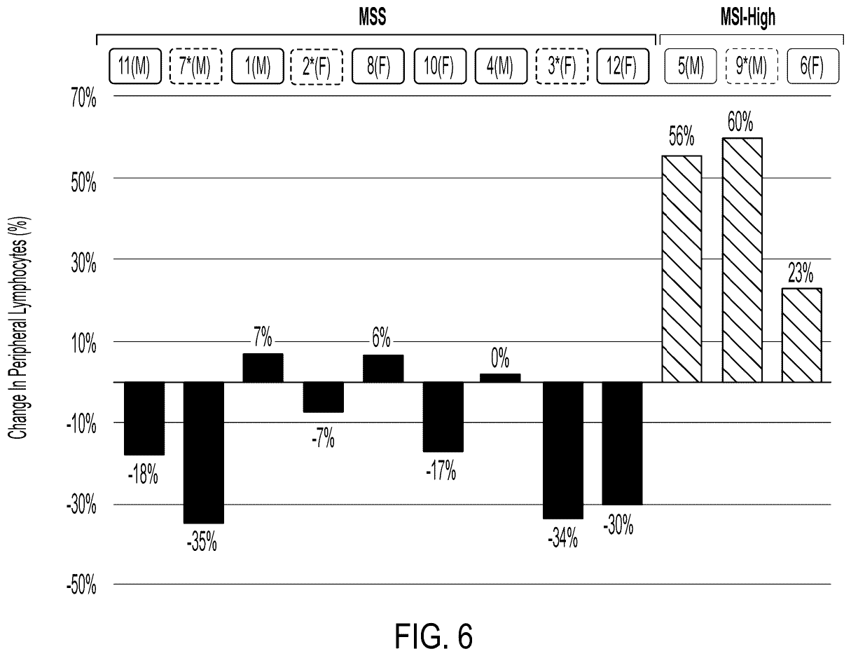

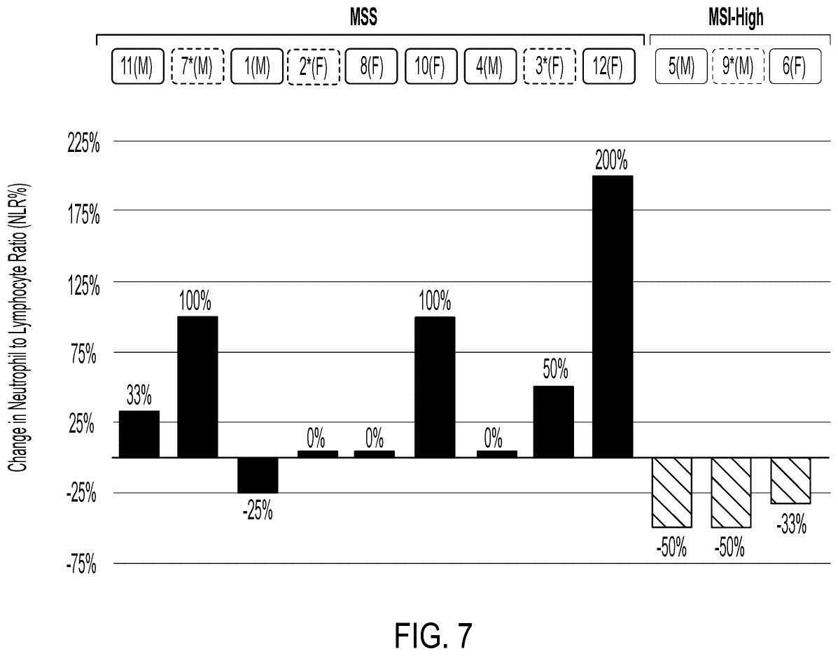

Circulatory immune cells kinetics in response to bot/bal were also assessed. Lymphocytes count were noted to decrease in most patients with pMMR/MSS tumors. Conversely, lymphocytes count increased in all patients with dMMR/MSI-H tumors (FIG. 6). The trend was mirrored by neutrophils as well, which increased in patients with pMMR/MSS tumors and decreased in patients with dMMR/MSI-H tumors. The changes in the neutrophil-to-lymphocyte-ratio (NLR) 2 weeks post bot/bal therapy is shown in FIG. 7.

### Radiological and Surgical Findings

The patient population was notable for a predominance of bulky stage III CRC on pre-treatment staging computed tomography (CT) and/or magnetic resonance imaging (MRI) scans (FIG. 2A). Only 4 out of 12 (33.3%) patients had imaging prior to surgery as it was not a requirement for the study. Clinically at the time of surgery there were significant reductions in the tumor size, with less luminal volume. In several cases there was still noted to be an inflammatory mass visible on CT scan, likely due to the infiltration of immune cells, showing ‘pseudo-progression’.

Of the 12 included patients, 8 had baseline CT only, 3 had baseline CT and MRI, and one had baseline CT and PET/CT. 3 had imaging findings suggestive of T2, NO disease and 9 had findings suggestive of T3, N+ disease. Four patients had follow-up imaging following neoadjuvant therapy and prior to surgery including one patient with PET showing suspected “pseudo-progression”, one patient with no radiologic evidence of disease after treatment, one patient with significant tumor regression, and one patient with extensive intraluminal tumor regression and changes in tumor signal suggesting treatment effect.

### Results for Cohorts A, B, and C

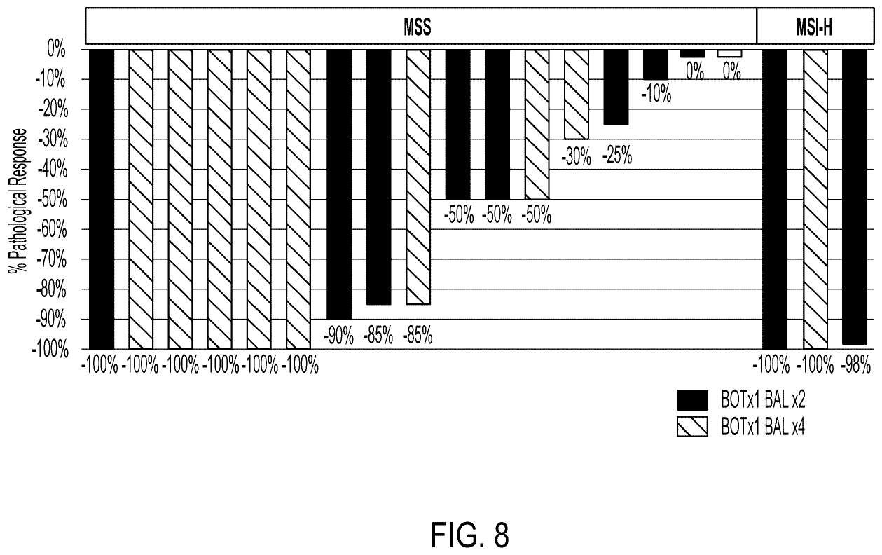

As of this time, results are available for 9 of the 13 cohort B patients and the 1 cohort C patient. The pathologic response (%) for the cohort B and cohort C patients is shown in FIG. 8. FIG. 8 also includes pathologic response for 10 of the 12 cohort A patients (2 of the rectal cancer patients were not included in this analysis). The patient demographics and safety data for the patients shown in FIG. 8 are summarized in Table 12, and the pathological response rates are summarized in Table 13.

TABLE 12
Patient demographics and safety results
according to treatment regimen.

| | BOTx1 BALx2 | BOTx1 BALx4 |
|---|---|---|
| | (n = 10) | (n = 10) |
| Median Age in | 67 (35-79) | 67 (23-76) |
| years (range) | | |
| Sex (F) | 70% | 40% |
| Median time to | 29.5 (21-38) | 57 (45-81) |
| surgery from | | |
| cycle 1 day 1 in | | |
| days (range) | | |
| Adjuvant | 70% | Too early to report |
| Chemotherapy | | |
| Unresolved | 0% | 0% |
| irAE* | | |

*2 patients with Grade 3 diarrhea/colitis managed with infliximab and short course steroids

TABLE 13
Pathological response rates according to treatment regimen.

| | |
|---|---|
| Pathological | Complete |
| Response | Pathological Response |
| (>50% Regression) | (100% Regression) |

BOTx1 BALx2 (n = 10)
(Cohort A)

| | | |
|---|---|---|
| pMMR/MSS (n = 8) | 5 (63%) | 1 (13%) |
| dMMR/MSI-H (n = 2) | 2 (100%) | 1 (50%) |

BOTx1 BALx4 (n = 10)

| | | |
|---|---|---|
| pMMR/MSS (n = 9) | 7 (78%) | 5 (56%) |
| (Cohort B) | | |
| dMMR/MSI-H (n = 1) | 1 (100%) | 1 (100%) |
| (Cohort C) | | |

Overall (n = 20)

| | | |
|---|---|---|
| Overall pMMR/MSS | 12 (71%) | 6 (35%) |
| (n = 17) | | |
| Overall dMMR/MSI-H | 3 (100%) | 2 (67%) |
| (n = 3) | | |

A deepening of pathologic response was seen in cohort B patients relative to cohort A. For the cohort B patients (pMMR/MSS), 7/9 (78%) had a ≥50% pathologic response. Of these, 5 (56%) had pathologic complete responses; 1 (11%) had 85% pathologic tumor reduction; and 1 (11%) had 50% pathologic tumor reduction. The two remaining cohort B patients had 30% and 0% pathologic tumor reduction. The 1 cohort C patient (dMMR/MSI-H) had a pathologic complete response. In summary, across all cohorts, 12/17 (71%) of patients with pMMR/MSS CRC had ≥50% pathologic responses and 3/3 (100%) of patients with dMMR/MSI-H CRC had major pathologic responses (≥90% pathologic reduction).

### Example 2. Further Analysis of Patient Tumors

This Example describes a more detailed analysis of tumor tissue pre- and post-treatment in two patients with pMMR/MSS colorectal cancer who displayed major pathological responses to the bot/bal regimen as described in Example 1. The analysis revealed an unprecedented ‘inside-out’ (serosal-to-mucosal) pattern of responses seen with the bot/bal regimen. The rapid immune response pattern observed has not been described previously in this setting. Spatial biology analyses revealed mechanisms of actions of bot, a novel innate/adaptive immune activator. These observations have downstream implications for neoadjuvant immunotherapy and potentially sparing patients chemotherapy.

The two cohort A patients received one fixed dose of 75 mg of bot and two doses of 240 mg of bal 2 weeks apart; with the first dose of bal the same day as the bot, as part of the study described in Example 1. Patients in the study could proceed to curative-intent surgical resection at least 1 week after the second dose of bal. Serial blood-based biomarkers including circulating tumor DNA (ctDNA) as well as tissue immune-microenvironment correlates were assessed both at baseline as well as in the surgical specimen. For the latter, a 13-marker immune-oncology panel was performed to test the pre- and post-treatment colon and rectal cancer samples in one staining round and one imaging round at 20× using a multimodal imaging instrument. The samples were analyzed via pathologist assessment and a quantitative image analysis pipeline for deep interrogation of the immune spatial environment. Pathology and quantitative image analysis included tumor immune cell subset percentages and tumor proliferation index, each of which was reviewed by a pathologist for tumor hot/cold assessment. In addition, cell proximity analysis was done to study interactions between up to four cell types by quantitative image analysis. Results were compiled and presented with descriptive statistics summarizing the intra- and inter-patient results and trends/patterns observed via both analysis methods.

The regression pattern seen in immuno-oncologic treatment of patients with lung cancer has been described as ‘outside-in’ (Cottrell et al., 2018, Ann. Oncol. 29(8):1853-1860). The ‘outside-in’ pattern of response infers the regression bed with the immune infiltrates typically surrounded the residual tumor foci and abutted normal background lung tissue. Conversely, as colon and rectal cancers develop inwards penetrating deeper layers of the colon wall and spreading to adjacent lymph nodes, the surprising and unexpected immunotherapy response described herein is typified by an ‘inside-out’ (serosa-to-mucosa) regression pattern.

The tumors analyzed were submitted in their entirety for histopathologic review. Microscopically, a dense mixed inflammatory infiltrate was identified surrounding the tumor mass. The infiltrate was lymphoplasmacytic-rich but also contained frequent macrophages (some foamy), occasional multinucleated giant cells, eosinophils, and neutrophils. Tertiary lymphoid structures (TLS) or Crohn like reaction was commonly seen at the periphery. The infiltrates were associated with patchy submucosal fibrosis, and one case showed frequent non-necrotizing granulomatous inflammation throughout the bowel wall. Neovascularization was a prominent feature in most tumor beds, and granulation tissue predominated along the luminal surface. Rather than viable tumor being haphazardly arranged within dense fibrosis, as is often seen with neoadjuvant chemotherapy, targeted therapy, or radiotherapy, viable tumor was often superficially oriented near the luminal surface within the tumor center, with dense inflammation surrounding the periphery and comprising most of the grossly identifiable tumor bed (i.e., regression bed as has been previously described). Residual tumor glands often demonstrated evidence of ongoing destruction with incomplete lumens and frequent luminal microabscesses.

The killing by immune cells is like a “wave” or “tsunami,” whereby the residual tumor is left at the tip/superficial layers of the colon in the mucosa/submucosa, and the deeper layers are spared. Both of the analyzed cases at outstart were advanced T-stage and node positive disease. Additionally, no residual cells were seen in deeper lymph nodes. Other high-risk features, for example, lymphovascular invasion (LVI), perineural invasion (PNI), or tumor budding were not seen. In other words, the cancer is being killed from the inside-out by the patient's own immune cells, which decreases the likelihood that any micrometastatic disease is left behind. From a mutational and next generation sequencing standpoint, there is nothing else to explain the exceptional responses. The first pMMR/MSS tumor was in the ascending colon, RAS-RAF-wildtype, TMB 6.4 Mut/Mb, and the second tumor was a high rectal cancer, KRAS-G12V mutant, and a TMB of 4.7. Final pathology in both cases was ypT1N0MX.

Analyses of the biopsy and surgical samples pre- and post-immunotherapy show a significant increase in and a diverse array of immune cells. Analyzed tissue sections from the first patient showed extensive expansion & infiltration of CD3+ T-cells confined to the tumor area in comparison to the adjacent normal colonic tissue. T-cells surrounded the tumor cells and extended deep into the muscle layer and the serosal layer. Crohn's-like reaction was present with many CD20+B-cells forming follicles specifically in the tumor area & extending into the deeper muscularis propria and serosa. Analysis of immune proliferation via Ki67 proliferation index showed a marked increase in a post-treatment surgical resection specimen (58%) compared to the pre-treatment biopsy specimen (23%), especially for T-cells. T-cell density (including all T-cell subsets) within the tumor increased dramatically with treatment (1398.4/mm² for resection vs 190.7/mm² for biopsy). Treg density also increased (411.0/mm² for resection vs 56.1/mm² for biopsy). The ratio of Treg to effector T helper cells in the tumor decreased (36% for resection vs 55% for biopsy).

Analysis of tissue sections from the second patient also showed an increase in immune cells. Extensive expansion and infiltration of CD3+ T-cells was seen in a tumor area (area #1) associated with tumor regression, in comparison to a second tumor area (area #2) which shows less extensive T-cell infiltration and no obvious tumor regression. CD4+T-helper cells were the main immune cell type in the tumor microenvironment (area #1: 1021.8/mm²; area #2: 389.3) in the post-treatment resection. Few T-helper cells were present superficially in the lamina propria of the adjacent normal colonic tissue. CD8 cytotoxic cells (CTLs) were more prevalent within the tumor area #1 (378.4/mm²) compared to tumor area #2 (134.2/mm²), consistent with tumor regression in the former but not in the latter. CTLs were markedly increased and clustered in the invasive margin (862.1/mm² and 604.1/mm² for areas #1 and #2, respectively) protecting the normal deeper tissue from invasion by tumor cells. Ki67 proliferation index analysis showed that immune cells were markedly increased in post-treatment surgical resection tumor area #2 (35%, mucosal adenocarcinoma) compared to area #1 (20%, deep invasive adenocarcinoma associated with tumor regression) and to the pre-treatment biopsy specimen (16%). T-cell density after treatment was increased within tumor area #1 (1267.7/mm², deep invasive adenocarcinoma associated with tumor regression) but not tumor area #2 (543.6/mm², mucosal adenocarcinoma) compared to the pre-treatment biopsy specimen (483.9/mm²). Treg density increased with treatment (236.5/mm² for area #1, 2013/mm² for area #2 vs 132.5/mm² for biopsy). The ratio of Treg to effector T helper cells in the tumor decreased for area #B and increased for area #2 (27e and 49s for resection vs 34% for biopsy).

Immune repertoire changes in both patients are comprehensively outlined in Table 14 and FIGS. 9A-9D and FIGS. 1A-10D. The pattern and trends were nearly identical in more than one instance. Of note, intra-tumoral microenvironment heterogeneity in terms of immune response was seen. For the second case, the zone of deeper regression was separated from the zone with not as much regression for analyses to outline some of the changes seen in these distinct areas. The values in Table 14 were calculated using PathViewer software. Briefly, a pathologist draws a region of interest (ROI) corresponding to tumor-bearing regions of the image, then counts cells based on visual inspection. The software reports ROI area, which allows calculation of cell density.

TABLE 14
Quantification of immune cells in patient tumors.

| Patient | Patient 1 | | Patient 2 | | |
|---|---|---|---|---|---|
| Treatment | Baseline | Surgical | Baseline | Surgical | Surgical |
| | Biopsy | Resection | Biopsy | Resection | Resection |
| Location | Ascending colon | | Sigmoid colon | | |
| Pathology | Tubular | Invasive | Invasive | Adenocarcinoma | Adenocarcinoma |
| | Adenoma | Adenocarcinoma | Adenocarcinoma | | |
| Area/Region | Total | Total tumor | Total tumor | Tumor area 1 | Tumor area 2 |
| of Interest | tumor | | | (deep invasive + | (mucosal) |
| (ROI) name | | | | ulcer) | |
| Area (mm²) | 12.06 | 6.01 | 5.321 | 10.57 | 14.9 |
| Analyte | Value | Value | Value | Value | Value |
| CD20+ cell | 2.2 | 161.4 | 28.2 | 132.5 | 30.2 |
| density | | | | | |
| CD68+ cell | 100.3 | 99.8 | 125.9 | 387.9 | 288.6 |
| density | | | | | |
| CD163+ | 106.6 | 73.2 | 138.1 | 170.3 | 255.0 |
| cell density | | | | | |
| CD3+ cell | 190.7 | 1389.4 | 483.9 | 1267.7 | 543.6 |
| density | | | | | |
| CD4+ cell | 126.0 | 1356.1 | 402.2 | 1021.8 | 389.3 |
| density | | | | | |
| CD8+ cell | 89.0 | 252.9 | 94.9 | 378.4 | 134.2 |
| density | | | | | |
| FOXP3+ | 56.1 | 411.0 | 132.5 | 236.5 | 201.3 |
| cell density | | | | | |
| FOXP3+ | 55% | 36% | 34% | 27% | 49% |
| fraction | | | | | |
| PD-1+ cell | 6.2 | 81.5 | 17.9 | 274.4 | 134.2 |
| density | | | | | |
| PD-L1 CPS | 58% | 70% | <1% | 10% | 30% |
| score | | | | | |
| PD-L1 TPS | <1% | 0% | 0% | 0% | <1% |
| score | | | | | |
| Immune | 23% | 58% | 16% | 20% | 35% |
| Proliferation | | | | | |
| Index | | | | | |

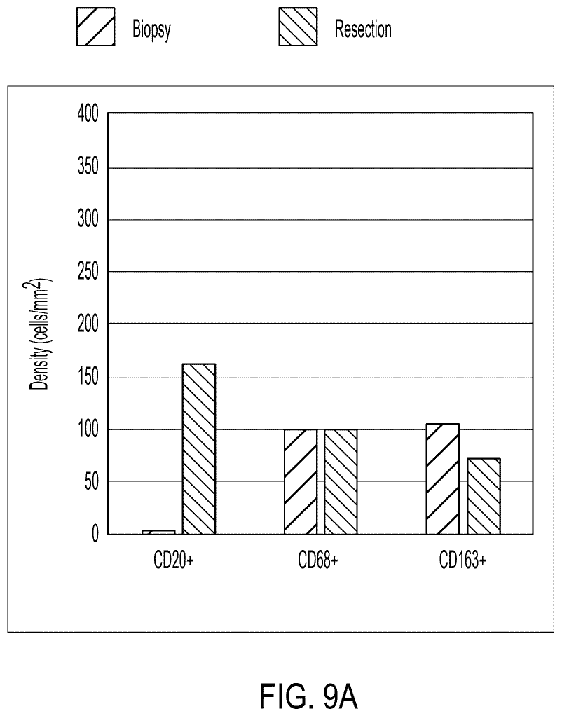

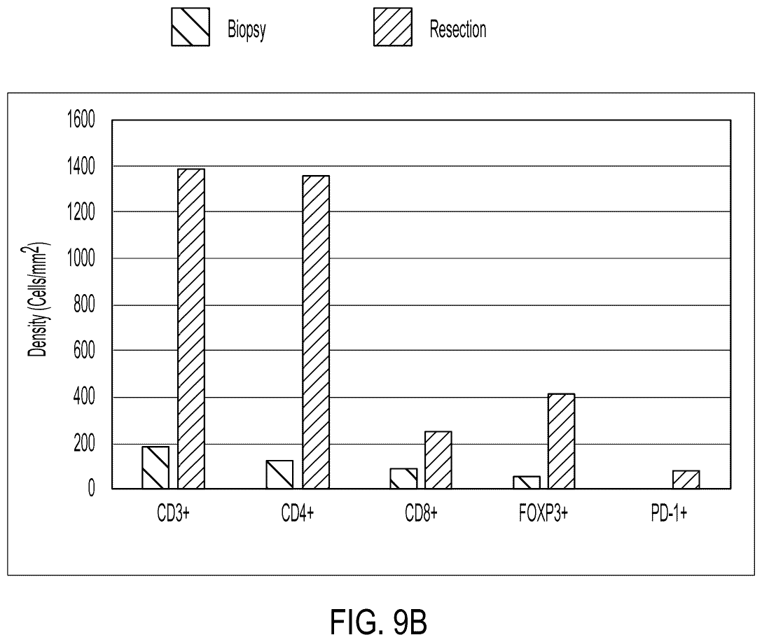

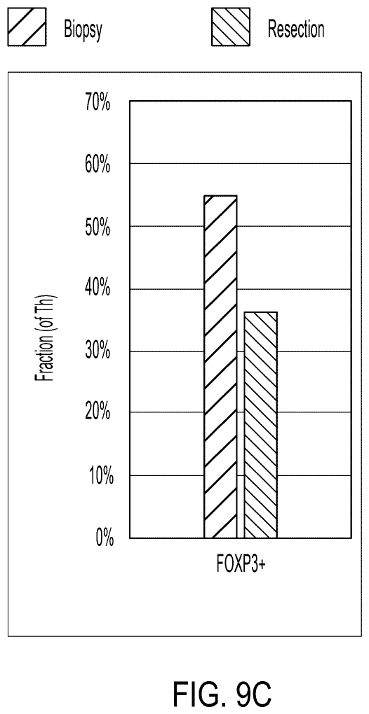

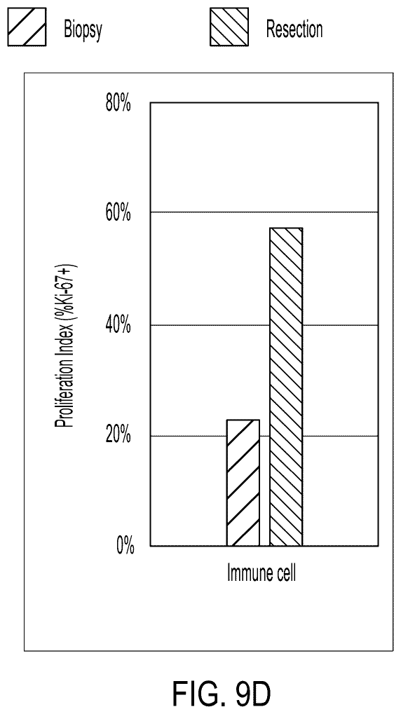

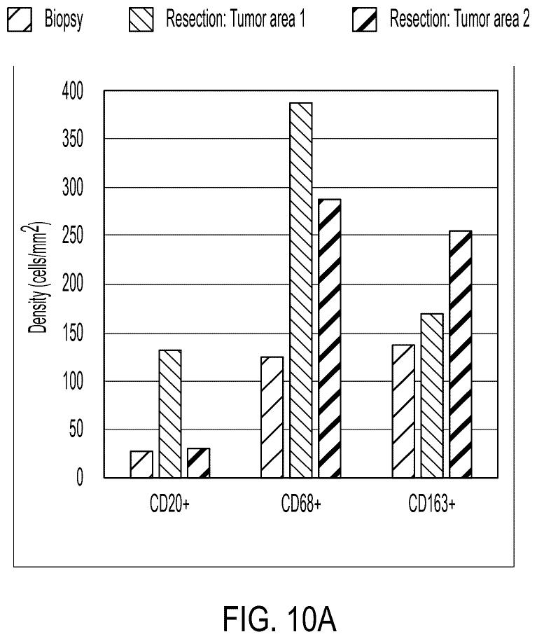

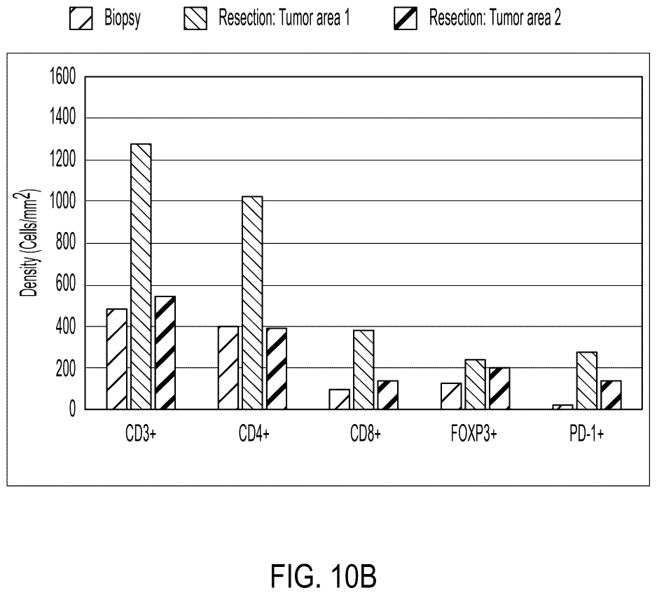

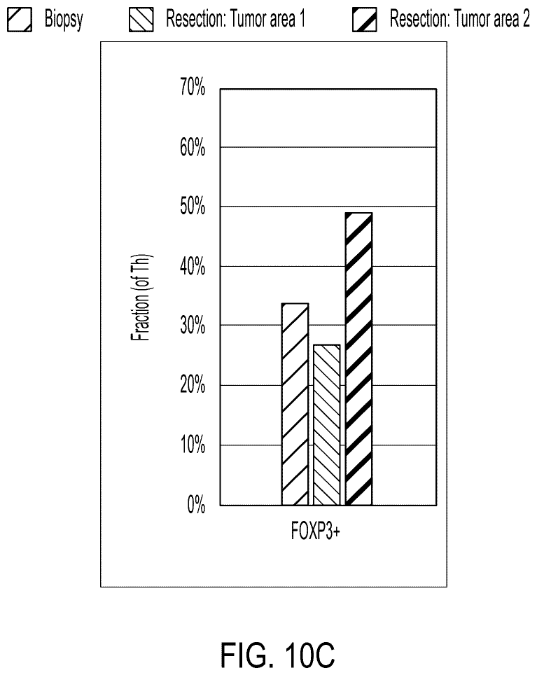

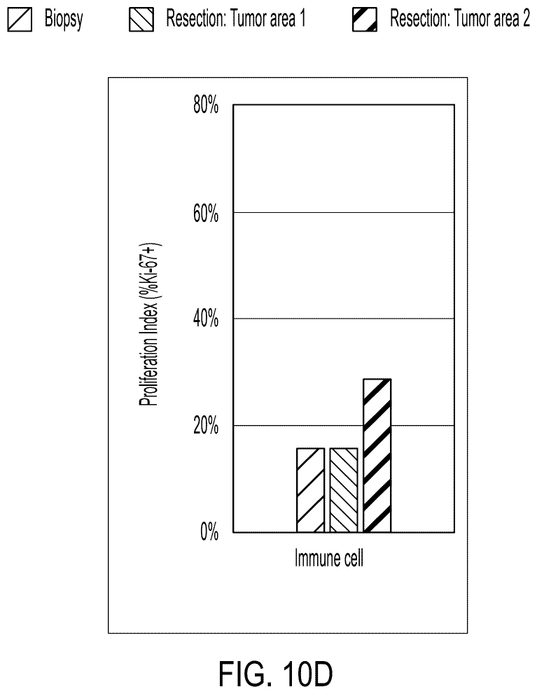

FIGS. 9A-9D and FIGS. 10A-10D show changes in immune cell populations in patients 1 and 2, respectively. For patient 1, all B and T-cell densities increased in the resection samples compared to the pre-treatment biopsy, while macrophage populations decreased. The proportion of Th cells that were Treg decreased in the resection samples compared to the pre-treatment biopsy, and the proportion of immune cells that were proliferating was increased in the resection samples compared to the pre-treatment biopsy. For patient 2, all B and T-cell densities increased in tumor area #1 of the resection samples compared to tumor area #2 and to the pre-treatment biopsy, while macrophage populations increased in both tumor areas upon treatment. The proportion of Th cells that were Treg decreased in tumor area #1 and increased in tumor area #2 of the resection samples compared to the pre-treatment biopsy, and the proportion of immune cells that were proliferating was increased in tumor area #2 of the resection samples compared to the pre-treatment biopsy.

This Example describes the first reported activity of the bot/bal regimen in patients with pMMR/MSS colon and rectal cancers in the neoadjuvant setting and shows a) unprecedented activity of an immunotherapeutic agent in pMMR/MSS colon and rectal tumors, and b) a pattern of regression and killing that has important clinical implications. If the pattern of response is consistent, i.e., all the tumor is left at the tip and regressed elsewhere in the whole surgical specimen, these patients could be spared the toxicity of adjuvant chemotherapy. The observation was consistently reported among the additional 10 patients described in Example 1, leading to the downstaging noted in FIG. 2A. The purpose of adjuvant therapy is to eradicate micrometastatic disease that might be left behind. Currently, for advanced colon and rectal cancer patients who undergo surgery and are noted to be node positive (stage III), 3 to 6 months of adjuvant chemotherapy is recommended. Neoadjuvant immunotherapy with this novel regimen could obviate the need for chemotherapy if it can eradicate micro-metastatic disease ‘inside-out’ since, even if there is residual disease on the final surgical specimen, it would be within the surgical resection specimen with potentially no micrometastases remaining.

### INCORPORATION BY REFERENCE

All patent and non-patent literature references cited above are incorporated herein by reference in their entirety.

1. A method of treating a colorectal tumor in a subject in need thereof, the method comprising administering to the subject:
a) a dose of a first antibody that specifically binds to human CTLA-4, wherein the first antibody comprises a human IgG heavy chain constant region that is a variant of a wild type human IgG heavy chain constant region, wherein the variant human IgG heavy chain constant region binds to FcγRIIIA with a higher affinity as compared to the affinity that the wild type human IgG heavy chain constant region binds to FcγRIIIA; and
b) a first dose of a human PD-1 inhibitor,
wherein the dose of the first antibody and the first dose of the human PD-1 inhibitor are each administered to the subject prior to surgical removal of the tumor.

2. The method of claim 1, further comprising administering to the subject:
c) a second dose of the human PD-1 inhibitor, wherein the second dose is administered to the subject prior to surgical removal of the tumor;
d) a third dose of the human PD-1 inhibitor, wherein the fourth dose is administered to the subject prior to surgical removal of the tumor; and/or
e) a fourth dose of the human-PD-1 inhibitor, wherein the fourth dose is administered to the subject prior to surgical removal of the tumor.

3. (canceled)

4. The method of claim 1, wherein the first antibody comprises:
a VH comprising the CDRH1, CDRH2, and CDRH3 amino acid sequences of the VH amino acid sequence set forth in SEQ ID NO: 7; and a VL comprising the CDRL1, CDRL2, and CDRL3 amino acid sequences of the VL amino acid sequence set forth in SEQ ID NO: 8;
the CDRH1, CDRH2, CDRH3, CDRL1, CDRL2, and CDRL3 amino acid sequences set forth in SEQ ID NOs: 1, 2, 3, 4, 5, and 6, respectively;
a VH comprising the amino acid sequence set forth in SEQ ID NO: 7 and a VL comprising the amino acid sequence set forth in SEQ ID NO: 8;
a human IgG1 heavy chain constant region comprising S239D/A330L/I332E mutations, numbered according to the EU numbering system; and/or
a heavy chain comprising the amino acid sequence set forth in SEQ ID NO: 9 and a light chain comprising the amino acid sequence set forth in SEQ ID NO: 10.

5.-8. (canceled)

9. The method of claim 1, wherein the first antibody is afucosylated.

10. The method of claim 1, wherein the first antibody is botensilimab.

11. The method of claim 1, wherein the human PD-1 inhibitor is a second antibody that specifically binds to human PD-1 or human PD-L1.

12. The method of claim 11, wherein the second antibody comprises:
a VH comprising the CDRH1, CDRH2, and CDRH3 amino acid sequences of the VH amino acid sequence set forth in SEQ ID NO: 17; and a VL comprising the CDRL1, CDRL2, and CDRL3 amino acid sequences of the VL amino acid sequence set forth in SEQ ID NO: 18;
the CDRH1, CDRH2, CDRH3, CDRL1, CDRL2, and CDRL3 amino acid sequences set forth in SEQ ID NOs: 11, 12, 13, 14, 15, and 16, respectively;
a VH comprising the amino acid sequence set forth in SEQ ID NO: 17 and a VL comprising the amino acid sequence set forth in SEQ ID NO: 18;
a human IgG4 heavy chain constant region comprising an S228P mutation, numbered according to the EU numbering system; and/or
a heavy chain comprising the amino acid sequence set forth in SEQ ID NO: 19 and a light chain comprising the amino acid sequence set forth in SEQ ID NO: 20.

13-16. (canceled)

17. The method of claim 11, wherein the second antibody is balstilimab, or is selected from the group consisting of: adebrelimab, atezolizumab, avelumab, camrelizumab, cemiplimab, cosibelimab, dostarlimab, durvalumab, enlonstobart, envafolimab, nivolumab, pembrolizumab, penpulimab, pidilizumab, prolgolimab, pucotenlimab, retifanlimab, serplulimab, sintilimab, socazolimab, sugemalimab, tagitanlimab, tislelizumab, toripalimab, and zimberelimab.

18. (canceled)

19. The method of claim 1, wherein:
the dose of the first antibody and the first dose of the human PD-1 inhibitor are administered on the same days;
the dose of the first antibody and the first dose of the human PD-1 inhibitor are administered simultaneously;
the dose of the first antibody is administered prior to the first dose of the human PD-1 inhibitor; and/or
the dose of the first antibody is administered after the first dose of the human PD-1 inhibitor.

20.-22. (canceled)

23. The method of claim 2, wherein the second dose of the human PD-1 inhibitor is administered 5 to 30 days, optionally 7 to 21 days, optionally 9 to 19 days, optionally 12 to 19 days, optionally 14 days after the first dose of the human PD-1 inhibitor is administered.

24. (canceled)

25. The method of claim 2, wherein the third dose of the human PD-1 inhibitor is administered 5 to 30 days, optionally 7 to 21 days, optionally 9 to 19 days, optionally 12 to 19 days, optionally 14 days after the second dose of the human PD-1 inhibitor is administered.

26. (canceled)

27. The method of claim 2, wherein the fourth dose of the human PD-1 inhibitor is administered 5 to 30 days, optionally 7 to 21 days, optionally 9 to 19 days, optionally 12 to 19 days, optionally 14 days after the third dose of the human PD-1 inhibitor is administered.

28. (canceled)

29. The method of claim 1, wherein the dose of the first antibody is about 10 mg to about 250 mg, about 25 mg to about 200 mg, about 50 mg to about 100 mg, or about 75 mg, and/or wherein the first, second, third, and/or fourth dose of the human PD-1 inhibitor is about 100 mg to about 750 mg, about 200 mg to about 500 mg, about 240 mg, or about 450 mg, optionally wherein:
the first, second, third, and fourth dose of the human PD-1 inhibitor is each about 240 mg;
the first, second, third, and/or fourth dose of the human PD-1 inhibitor is about 450 mg; and/or
the first, second, third, and fourth dose of the human PD-1 inhibitor is each about 450 mg.

30.-35. (canceled)

36. The method of claim 2, wherein the dose of the first antibody and/or the first dose of the human PD-1 inhibitor is administered 10 to 100 days, optionally 14 to 98 days, optionally 14 to 91 days, optionally 14 to 84 days, optionally 14 to 77 days, optionally 14 to 70 days, optionally 14 to 63 days, optionally 14 to 56 days, optionally 16 to 61 days, optionally 19 to 61 days, optionally 21 to 56 days before surgical removal of the tumor, optionally wherein:
the dose of the first antibody and/or the first dose of the human PD-1 inhibitor is administered 28 days before surgical removal of the tumor;
the dose of the first antibody and/or the first dose of the human PD-1 inhibitor is administered 56 days before surgical removal of the tumor;
the second dose of the human PD-1 inhibitor is administered 1 to 60 days, optionally 5 to 45 days, optionally 7 to 42 days before surgical removal of the tumor;
the second dose of the human PD-1 inhibitor is administered at least 7 days before surgical removal of the tumor;
the second dose of the human PD-1 inhibitor is administered 14 days before surgical removal of the tumor;
the fourth dose of the human PD-1 inhibitor is administered 1 to 60 days, optionally 5 to 45 days, optionally 7 to 42 days before surgical removal of the tumor;
the fourth dose of the human PD-1 inhibitor is administered at least 7 days before surgical removal of the tumor; and/or
the fourth dose of the human PD-1 inhibitor is administered 14 days before surgical removal of the tumor.

37.-44. (canceled)

45. The method of claim 1, wherein the colorectal tumor is a primary tumor, a colorectal adenocarcinoma, and/or is not metastatic, optionally wherein:
the colorectal tumor is microsatellite instable—high (MSI-H);
the colorectal tumor is not microsatellite instable—high (MSI-H);
the colorectal tumor is microsatellite stable (MSS);
the colorectal tumor is mismatch repair deficient (dMMR); or
the colorectal tumor is not mismatch repair deficient (dMMR).

46.-47. (canceled)

48. The method of am claim 1, wherein the subject has a RAS mutation, optionally wherein the RAS mutation is a KRAS or NRAS mutation: wherein the subject is at least 18 years of age; and/or wherein the subject has histologically, cytologically, or clinically confirmed adenocarcinoma of the colon.

49.-54. (canceled)

55. The method of claim 1, wherein the first antibody and/or the human PD-1 inhibitor is administered intravenously; and/or wherein the first antibody and/or the human PD-1 inhibitor is administered by intravenous infusion over about 30 minutes.

56.-58. (canceled)

59. The method of claim 1, wherein before administration of the first antibody and/or the human PD-1 inhibitor:
the subject has an Eastern Cooperative Oncology Group performance status of 0-2;
the subject has adequate organ and bone marrow reserve function as defined by one or more of:
a) absolute neutrophil count≥1.5×10⁹ per L;
b) platelets≥100×10⁹ per L;
c) hemoglobin≥8.0 g/dL without a transfusion that has occurred within 2 weeks of hemoglobin measurement;
d) creatinine clearance≥40 mL/min as measured or calculated per local institutional standards;
e) aspartate aminotransferase≤2.5× upper limit of normal (ULN);
f) alanine aminotransferase≤2.5×ULN; and
g) total bilirubin≤1.5×ULN;
the subject does not have metastases identified using standard of care radiographic imaging;
the subject is not pregnant and/or is not breastfeeding;
the subject does not have clinically significant cardiovascular disease or an active infection requiring treatment.

60.-62. (canceled)

63. The method of claim 1, wherein the subject has not received a live vaccination within 28 days prior to administration of the first antibody and/or the human PD-1 inhibitor, and/or the subject has not received systemic corticosteroid therapy within 7 days prior to administration of the first antibody and/or the human PD-1 inhibitor.

64.-65. (canceled)

66. The method of claim 1, wherein administration of the first antibody and the human PD-1 inhibitor:
reduces residual viable tumor cells in the subject following surgical removal of the tumor;
reduces minimal residual disease in the subject following surgical removal of the tumor, optionally wherein minimal residual disease is assessed via measurement of circulating tumor DNA;
reduces the size of the tumor in the subject;
increases T-cell, memory T-cell, myeloid cell, and/or antigen presenting cell activation in the subject; and/or
reduces the number of Treg cells in the subject.

67.-74. (canceled)
